SEQUENCE LISTING (26 sequences)

SEQ ID NO: 1 - protein, 732 aa, Artificial Sequence
  DMAb-2G4
MDWTWRILFLVAAATGTHAEVQLQESGGGLMQPGGSMKLSCVASGFTFSNYWMNWVRQSPEKGLEWVAEIRLKSNNYATHYAESVKGRFTISRDDSKRSVYLQMNTLRAEDTGIYYCTRGNGNYRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPASLSVSVGETVSITCRASENIYSSLAWYQQKQGKSPQLLVYSATILADGVPSRFSGSGSGTQYSLKINSLQSEDFGTYYCQHFWGTPYTFGGGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 2 - DNA, 2202 bp, Artificial Sequence
  PGX9226, DMAb-2G4
atggattggacatggaggattctgtttctggtcgccgccgcaactggaacccacgctgaagtgcagctgcaggagtcaggaggaggactgatgcagcccggcggaagcatgaagctgtcctgcgtggcatctggcttcacctttagtaactactggatgaattgggtccgccagtcacctgagaagggactggaatgggtggctgagatccgactgaaaagcaacaattacgccacccactatgctgagtcagtgaaggggcgattcacaattagcagggacgattctaaaagaagtgtgtatctgcagatgaacactctgagagccgaagacaccggaatctactattgcacacggggcaacggaaattaccgcgctatggattattgggggcagggcacttccgtcaccgtgagctccgcaagcacaaagggaccctccgtgtttcccctggccccttctagtaaaagcacctccggaggaacagcagctctgggatgtctggtgaaggactacttccctgagccagtcaccgtgtcatggaacagcggagccctgacctctggggtccatacatttcctgctgtgctgcagtcaagcgggctgtactccctgtcctctgtggtcactgtgccaagttcaagcctgggcactcagacctatatctgcaacgtgaatcacaagcccagcaataccaaagtcgacaagaaagtggagcctaagtcctgtgataaaacacatacttgcccaccttgtccagcacctgaactgctgggaggacctagcgtgttcctgtttccacccaagccaaaagacacactgatgatttcccgcactcctgaggtcacctgtgtggtcgtggacgtgtctcacgaggaccccgaagtcaagttcaactggtacgtggatggcgtcgaagtgcataatgccaagaccaaacccagggaggaacagtacaactctacctatagggtcgtgagtgtcctgacagtgctgcaccaggactggctgaacggcaaggagtataagtgcaaagtgagcaataaggctctgccagcacccatcgaaaaaactatttccaaggccaaaggacagccaagagagccccaggtgtacaccctgcctccatctcgggacgaactgacaaagaaccaggtcagtctgacttgtctggtgaaaggcttctatccatccgatatcgctgtggagtgggaatctaatggacagcccgagaacaattacaagaccacaccccctgtgctggactccgatgggtctttctttctgtatagtaagctgaccgtggataaatcacggtggcagcagggcaacgtcttttcttgcagtgtgatgcatgaagccctgcacaatcattacacacagaagtcactgagcctgtccccaggcaagcgaggacgaaaaaggagatctggaagtggggctactaacttcagcctgctgaaacaggcaggcgacgtggaggaaaatcctggaccaatggtcctgcagacccaggtgtttatctcactgctgctgtggattagcggggcttacggcgatattcagatgacacagtccccagcatcactgagcgtctccgtgggagaaacagtgtccatcacttgtcgcgcctctgagaacatctacagcagcctggcttggtatcagcagaagcagggaaaaagcccccagctgctggtctactccgcaacaatcctggccgacggggtgccttctaggttctctggcagtggatcagggacacagtatagcctgaagattaatagtctgcagtcagaggattttgggacttactattgccagcacttctggggcacaccatacacttttggcggagggactaagctggagatcaaaaccgtcgcagccccctctgtgttcatttttccacccagtgacgaacagctgaagagtggcaccgcctcagtcgtgtgtctgctgaacaatttctaccctagagaggcaaaggtccagtggaaagtggataacgccctgcagagcggcaattcccaggaatctgtgactgagcaggacagtaaggattcaacctatagcctgtccaacaccctgacactgagcaaagctgactacgaaaagcacaaagtctatgcatgcgaggtgacacatcagggactgagttcaccagtgactaagtcctttaatcggggggagtgttgataa

SEQ ID NO: 3 - protein, 711 aa, Artificial Sequence
  DMAb-4G7
QVQLQQSGPELEMPGASVKISCKASGSSFTGFSMNWVKQSNGKSLEWIGNIDTYYGGTTYNQKFKGKATLTVDKSSSTAYMQLKSLTSEDSAVYYCARSAYYGSTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPASLSASVGETVTITCRASENIYSYLAWYQQKQGKSPQLLVYNAKTLIEGVPSRFSGSGSGTQFSLKINSLQPEDFGSYFCQHHFGTPFTFGSGTELEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 4 - DNA, 2196 bp, Artificial Sequence
  pGX9229, DMAb-4G7
atggattggacatggaggattctgtttctggtcgccgccgccactggaacccacgcccaggtgcagctgcagcagtcagggcctgagctggaaatgcctggcgcttctgtgaaaatcagttgcaaggcatcaggaagctccttcacagggtttagcatgaactgggtgaaacagagcaatgggaagtccctggagtggatcggcaacattgacacctactatggcggaaccacatacaatcagaagttcaaaggcaaggctacactgactgtggacaaatctagttcaaccgcatatatgcagctgaagagcctgacatccgaggattctgcagtgtactattgcgctagatcagcatactatggcagcactttcgcctactggggccagggaaccctggtcacagtgagctccgcctccaccaaaggaccatctgtgtttcccctggctccttctagtaagagtacatcaggaggaactgcagctctgggatgtctggtgaaggattatttccctgagccagtcaccgtgagttggaactcaggcgcactgacttctggagtccacacctttcctgccgtgctgcagtcaagcggcctgtacagcctgtcctctgtggtcaccgtgccaagttcaagcctgggaacccagacatatatctgcaacgtgaatcacaaaccctctaatacaaaggtcgacaagaaagtggaacctaaaagctgtgataagactcatacctgcccaccttgtccagcacctgagctgctgggagggccttccgtgttcctgtttccacccaaaccaaaggacacactgatgattagcagaacccctgaagtcacatgtgtggtcgtggacgtgtcccacgaggaccccgaagtcaagttcaactggtacgtggatggcgtcgaggtgcataatgctaaaaccaagccccgagaggaacagtacaactctacttatagggtcgtgagtgtcctgaccgtgctgcaccaggactggctgaacggcaaggagtataaatgcaaggtgtctaacaaggccctgccagctcccatcgagaagacaattagcaaagctaagggacagccaagagaaccccaggtgtacactctgcctccatctcgggacgagctgaccaaaaaccaggtcagtctgacatgtctggtgaagggattctatccaagcgatatcgcagtggagtgggaatccaatgggcagcccgaaaacaattacaagactaccccccctgtgctggacagcgatggcagcttcttcctgtattccaaactgacagtggataagtctcggtggcagcaggggaacgtctttagctgctccgtgatgcatgaggccctgcacaatcattacactcagaagtctctgagtctgtcaccaggcaaacgaggacgaaagaggagaagcgggtccggagcaaccaacttctccctgctgaagcaggctggagacgtggaggaaaatcctgggccaatggtcctgcagacacaggtgtttatctcactgctgctgtggattagcggggcctacggcgatattcagatgactcagagccccgcatctctgagtgcctcagtcggcgagacagtgactatcacctgtcgcgcaagtgaaaacatctactcatatctggcctggtaccagcagaaacaggggaagagcccccagctgctggtctataatgctaaaaccctgatcgaaggagtgccttcccgattcagcggcagcgggtctggcacacagtttagcctgaagattaactccctgcagccagaggacttcggcagctacttttgccagcaccatttcggaactcccttcacctttggcagcgggacagagctggaaatcaaaactgtcgcagcccccagtgtgttcatttttccaccctcagacgaacagctgaagtctgggaccgccagtgtcgtgtgtctgctgaacaatttttaccctcgggaggctaaagtccagtggaaggtggataacgcactgcagtctggaaatagtcaggagtcagtgacagaacaggacagcaaagattccacttatagtctgtcaaacacactgactctgtctaaggccgactacgagaaacacaaggtctatgcttgcgaagtgactcatcaggggctgtcctctcctgtgaccaagagcttcaatcgcggcgagtgttgataa

SEQ ID NO: 5 - protein, 733 aa, Artificial Sequence
  DMAB4
MDWTWRILFLVAAATGTHAQVTLKESGPGILKPSQTLSLTCSLSGFSLSTSGVGVGWFRQPSGKGLEWLALIWWDDDKYYNPSLKSQLSISKDFSRNQVFLKISNVDIADTATYYCARRDPFGYDNAMGYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSQKFMSTSVGDRVSLTCKASQNVGTAVAWYQQKPGQSPKLLIYSASNRYTGVPDRFTGSGSGTDFTLTISNMQSEDLADYFCQQYSSYPLTFGAGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 6 - DNA, 2226 bp, Artificial Sequence
  pGX9230, DMAb4
ggatccgccgccaccatggactggacttggagaattctgttcctggtcgcagcagccactgggacacacgcacaggtgacactgaaagagagcggacccggaatcctgaaaccaagccagactctgtccctgacctgcagcctgtccggcttctctctgagtacctcaggagtcggagtgggatggtttcgacagccaagcggaaagggactggagtggctggccctgatctggtgggacgatgacaagtactataacccttcactgaaaagccagctgagcatttccaaggatttctctcgcaaccaggtctttctgaagatcagtaatgtggatattgccgacactgctacctactattgcgctaggagagatccattcggctacgacaatgcaatgggatattggggccagggaacctccgtcacagtgagctccgcatccacaaaagggccctctgtgtttcccctggccccttctagtaagtctacaagtggcggaactgccgctctgggctgtctggtgaaggactacttccctgagccagtcaccgtgtcctggaactctggagccctgacttctggggtccacacctttcctgctgtgctgcagtcaagcggactgtacagcctgtcctctgtggtcaccgtgccaagttcaagcctggggacacagacttatatctgcaacgtgaatcacaagccatctaatacaaaagtcgataagaaagtggaacccaagagctgtgacaaaacccatacatgcccaccttgtccagcacctgagctgctgggaggaccaagcgtgttcctgtttccacccaagcctaaagatacactgatgatttccaggacccccgaagtgacatgtgtggtcgtggatgtgtctcacgaggaccctgaagtcaagttcaactggtacgtggacggcgtcgaggtgcataatgctaagaccaaacctcgcgaggaacagtacaacagtacatatcgagtcgtgtcagtgctgaccgtcctgcaccaggactggctgaacggaaaggagtataagtgcaaagtgagcaacaaggcactgccagcccccatcgagaagactatttccaaggcaaaagggcagccaagggaaccccaggtgtacaccctgcctccaagcagagatgagctgactaaaaaccaggtctccctgacctgtctggtgaaggggttctatcctagtgacatcgctgtggagtgggaatcaaatggccagccagaaaacaattacaagaccacaccccctgtgctggatagtgacggctcattctttctgtattcaaagctgaccgtggataaaagcagatggcagcagggaaacgtcttctcatgcagcgtgatgcatgaggccctgcacaatcattacactcagaaatccctgtctctgagtcccggcaagcgaggaaggaaacggcgctcagggagcggcgctacaaacttttccctgctgaagcaggcaggggacgtggaggaaaatcctggcccaatggtcctgcagacccaggtgttcatcagcctgctgctgtggatttccggggcctacggcgatattgtgatgacccagagccagaagttcatgtccacatctgtcggcgaccgggtgtctctgacctgtaaggccagtcagaacgtcggaactgctgtggcatggtatcagcagaagcctgggcagtccccaaaactgctgatctacagtgcttcaaacagatataccggcgtgcctgatcggttcaccggaagcgggtccggcacagactttactctgaccatttctaatatgcagagtgaagatctggctgactacttctgccagcagtactcctcttatccactgacatttggagcagggactaagctggaaatcaaaacagtcgcagccccctccgtgttcatttttccaccctctgatgagcagctgaagtcaggcactgccagcgtcgtgtgtctgctgaacaatttctaccccagggaggccaaggtccagtggaaagtggacaacgctctgcagagcggaaattcccaggagtctgtgactgaacaggatagtaaagactcaacctattctctgagtaacacactgactctgtccaaggcagactacgagaagcacaaagtctatgcctgcgaagtgacccatcagggcctgagttcaccagtgacaaagtcttttaatcgcggagagtgttgataactcgag

SEQ ID NO: 7 - protein, 729 aa, Artificial Sequence
  DMAb-10
MDWTWRILFLVAAATGTHAEVQLVESGGGLVQPGGSLRLSCAASGFTFRSYDMHWVRQATGKGLEWVSAIGTAGDTYYPGSVKGRFTISRENAKNSLYLQMNSLRAEDTAVYYCARVRFGDTAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDRVTITCRASQSISSFLNWHQQKPGKAPKLLIYAASSLQSGVPSRFSGSGSGTDFTLTISSLQPEDFAIYYCQQSYISPFTFGPGTKVDIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 2214 bp, Artificial Sequence
  pGX9244, DMAb-10
ggatccgccgccaccatggactggacttggagaatcctgttcctggtcgccgccgctactgggactcatgccgaggtgcagctggtcgaatctggagggggcctggtgcagcctggcggcagcctgaggctgtcctgcgcagcatctggcttcacctttaggagctacgacatgcactgggtgcgccaggcaacaggcaagggactggagtgggtgtctgccatcggaaccgcaggcgatacatactatccaggctccgtgaagggcaggttcaccatctcccgcgagaacgccaagaattctctgtacctgcagatgaacagcctgagagccgaggacaccgccgtgtactattgcgccagggtgcgcttcggcgacacagcagtggattattggggccagggcaccctggtgacagtgagctccgcctccaccaagggaccaagcgtgttcccactggcaccttctagcaagtccacctctggcggcacagccgccctgggctgtctggtgaaggattacttccctgagccagtgacagtgtcctggaactctggcgccctgaccagcggagtgcacacatttcctgccgtgctgcagtcctctggcctgtactccctgagctccgtggtgaccgtgccatctagctccctgggcacccagacatatatctgcaacgtgaatcacaagcctagcaatacaaaggtggacaagaaggtggagccaaagtcctgtgataagacccacacatgccctccctgtccagcacctgagctgctgggcggcccaagcgtgttcctgtttccacccaagcccaaggacaccctgatgatcagccggaccccagaggtgacatgcgtggtggtggacgtgtcccacgaggaccccgaggtgaagtttaactggtacgtggatggcgtggaggtgcacaatgccaagaccaagccccgggaggagcagtacaacagcacctatagagtggtgtccgtgctgacagtgctgcaccaggactggctgaacggcaaggagtataagtgcaaggtgagcaataaggccctgccagcccccatcgagaagaccatctccaaggcaaagggacagccaagggagccacaggtgtacacactgcctccatcccgcgacgagctgaccaagaaccaggtgtctctgacatgtctggtgaagggcttctatccctctgatatcgccgtggagtgggagagcaatggccagcctgagaacaattacaagaccacaccccctgtgctggacagcgatggctccttctttctgtattccaagctgaccgtggacaagtctcggtggcagcagggcaacgtgtttagctgctccgtgatgcacgaggccctgcacaatcactacacccagaagtctctgagcctgtccccaggcaagaggggaagaaagcggagatctggcagcggcgccacaaacttcagcctgctgaagcaggccggcgatgtggaggagaatcctggcccaatggtgctgcagacccaggtgtttatctctctgctgctgtggatcagcggcgcctatggcgacatccagatgacacagtccccttctagcctgtccgcctctgtgggcgatcgggtgaccatcacatgtagagccagccagtccatctcctctttcctgaactggcaccagcagaagcctggcaaggccccaaagctgctgatctacgcagccagctccctgcagagcggagtgccctccaggttctctggcagcggctccggaaccgactttaccctgacaatctctagcctgcagcctgaggattttgccatctactattgccagcagtcttatatcagccccttcacctttggccctggcacaaaggtggacatcaagaccgtggccgccccaagcgtgttcatctttccaccctccgatgagcagctgaagtctggcacagccagcgtggtgtgcctgctgaacaatttctacccccgcgaggccaaggtgcagtggaaggtggacaacgccctgcagtccggcaattctcaggagagcgtgaccgagcaggactccaaggattctacatattctctgagctctaccctgacactgagcaaggccgattacgagaagcacaaggtgtatgcctgcgaggtcacccaccaggggctgtcaagtccagtcactaagtccttcaatcggggcgaatgctgataactcgag

SEQ ID NO: 9 - protein, 739 aa, Artificial Sequence
  DMAb-11
MDWTWRILFLVAAATGTHAEVQLVESGGGLIQPGGSLRLSCAASGFAVRSNYLSWVRQAPGKGLEWVSLIYSGGLTAYADSVEGRFTISRDNSKNTLYLQMNSLRVEDTALYYCARVASSAGTFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPRSLSVTPGEPASISCRSSQSLLHRNGYNYLDWYLQKPGQSPQLLIYLGSNRASGVPDRFSGSGSGTDFTLKISRVEAEDVGVYYCMQALQTPSWTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 2244 bp, Artificial Sequence
  pGX9256, DMAb-11
ggatccgccgccaccatggactggacctggagaatcctgttcctggtggcagcagcaaccggaacacacgcagaggtgcagctggtggagagcggcggcggcctgatccagccaggcggcagcctgaggctgtcctgcgcagcatctggatttgccgtgaggagcaactacctgtcctgggtgagacaggcaccaggcaagggactggagtgggtgtctctgatctacagcggcggcctgaccgcatatgcagacagcgtggagggcaggttcaccatctccagagataactctaagaatacactgtatctgcagatgaattccctgcgggtggaggacaccgccctgtactattgcgcccgcgtggccagctccgccggcacattctactatggcatggacgtgtggggccagggcaccacagtgaccgtgtctagcgcctccacaaagggaccaagcgtgttcccactggcaccttcctctaagtccacctctggcggcacagccgccctgggctgtctggtgaaggattacttccctgagccagtgaccgtgtcttggaacagcggcgccctgaccagcggagtgcacacatttcctgccgtgctgcagagctccggcctgtactccctgtctagcgtggtgaccgtgccatcctctagcctgggcacccagacatatatctgcaacgtgaatcacaagcctagcaatacaaaggtggacaagaaggtggagccaaagtcctgtgataagacccacacatgccctccctgtccagcacctgagctgctgggcggcccaagcgtgttcctgtttccacccaagcccaaggacacactgatgatctctaggaccccagaggtgacatgcgtggtggtggacgtgagccacgaggaccccgaggtgaagtttaactggtacgtggatggcgtggaggtgcacaatgccaagaccaagccaagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccaagggagccacaggtgtacacactgcctccatccagagacgagctgaccaagaaccaggtgtctctgacatgtctggtgaagggcttctatccctctgatatcgccgtggagtgggagagcaatggccagcctgagaacaattacaagaccacaccccctgtgctggactccgatggctctttctttctgtattccaagctgaccgtggataagtctcggtggcagcagggcaacgtgtttagctgctccgtgatgcacgaggccctgcacaatcactacacccagaagtctctgagcctgtcccctggcaagaggggaaggaagaggagatctggcagcggcgccacaaacttcagcctgctgaagcaggcaggcgacgtggaggagaatcctggaccaatggtgctgcagacccaggtgtttatctctctgctgctgtggatcagcggcgcctacggcgatatcgtgatgacccagtcccctcgctccctgtctgtgacacctggcgagccagccagcatctcctgtcggtcctctcagtctctgctgcaccgcaacggctacaattatctggactggtacctgcagaagcccggccagtcccctcagctgctgatctatctgggcagcaacagggcatccggagtgccagaccgcttctctggcagcggctccggaaccgacttcaccctgaagatcagcagggtggaggccgaggatgtgggcgtgtactattgcatgcaggccctgcagaccccctcctggacattcggccagggcaccaaggtggagatcaagacagtggccgcccctagcgtgttcatctttccaccctccgacgagcagctgaagtctggcaccgccagcgtggtgtgcctgctgaacaacttctaccccagagaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtgacccaccagggcctgtctagccctgtgacaaagtccttcaacagaggcgagtgttgataactcgag

SEQ ID NO: 11 - protein, 741 aa, Artificial Sequence
  DMAb12
MDWTWRILFLVAAATGTHAQVQLVQSGAEVKKPGSSVKVSCKASGATFGSDTVTWVRQAPGQGLEWMGGIIPFFGEANYAQRFQGRVTITADKSTNTAYMELSSLRSEDTAVYFCARQINEMATFGEIHYYTYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGGSELTQDPAVSVALGQTVRITCQGDSLRNYYASWYQQKPRQAPVLVFYGKNNRPSGIPDRFSGSSSGNTASLTISGAQAEDEADYYCNSRDSSSNHLVFGGGTKLTVLSTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 12 - DNA, 2232 bp, Artificial Sequence
  pGX9260, DMAb12
catggactggacctggagaatcctgttcctggtggcagcagcaaccggaacacacgcacaggtgcagctggtgcagagcggagcagaggtgaagaagccaggcagctccgtgaaggtgtcctgcaaggcatctggagccaccttcggctccgataccgtgacatgggtgcgccaggcaccaggacagggactggagtggatgggcggcatcatccctttctttggcgaggccaactacgcccagcggtttcagggcagagtgaccatcacagccgacaagtccaccaatacagcctacatggagctgtctagcctgcggtctgaggataccgccgtgtatttctgcgccagacagatcaacgagatggccacctttggcgagatccactactatacatacatggacgtgtggggacagggcaccctggtgacagtgtcctctgcctccaccaagggacctagcgtgttcccactggcacctagctccaagtctaccagcggcggcacagccgccctgggatgtctggtgaaggattatttccctgagccagtgacagtgtcctggaactctggcgccctgaccagcggagtgcacacatttcccgccgtgctgcagtctagcggcctgtactccctgtcctctgtggtgaccgtgcctagctcctctctgggcacccagacatatatctgcaacgtgaatcacaagccttctaatacaaaggtggacaagaaggtggagccaaagagctgtgataagacccacacatgccctccctgtccagcacctgagctgctgggcggcccaagcgtgttcctgtttccacccaagcccaaggacaccctgatgatcagcaggacccctgaggtgacatgcgtggtggtggacgtgtcccacgaggaccccgaggtgaagttcaactggtacgtggatggcgtggaggtgcacaatgccaagaccaagccccgggaggagcagtacaacagcacctatagagtggtgtccgtgctgacagtgctgcaccaggactggctgaacggcaaggagtataagtgcaaggtgtctaataaggccctgccagcccccatcgagaagaccatctccaaggcaaagggacagccaagggagccacaggtgtacacactgcctccaagccgcgacgagctgaccaagaaccaggtgtccctgacatgtctggtgaagggcttctatccatccgatatcgccgtggagtgggagtctaatggccagcccgagaacaattacaagaccacaccccctgtgctggacagcgatggctccttctttctgtattccaagctgaccgtggacaagtctcggtggcagcagggcaacgtgttttcctgctctgtgatgcacgaggccctgcacaatcactacacccagaagagcctgtccctgtctcctggcaagaggggaaggaagcggagaagcggctccggagccacaaacttcagcctgctgaagcaggccggcgatgtggaggagaatcctggcccaatggtgctgcagacccaggtgtttatctctctgctgctgtggatcagcggagcatacggcggctccgagctgacacaggacccagccgtgagcgtggccctgggacagaccgtgaggatcacatgtcagggcgatagcctgcgcaactactatgcctcctggtaccagcagaagcctcggcaggccccagtgctggtgttctatggcaagaacaataggccctctggcatccctgaccgctttagcggcagctcctctggcaataccgcaagcctgacaatctccggagcacaggcagaggacgaggcagattactattgcaacagcagagatagctcctctaatcacctggtgttcggcggcggaaccaagctgacagtgctgtctaccgtggccgccccaagcgtgttcatctttccaccctccgacgagcagctgaagtctggcacagccagcgtggtgtgcctgctgaacaacttctacccccgggaggccaaggtgcagtggaaggtggataacgccctgcagtctggcaatagccaggagtccgtgaccgagcaggactctaaggatagcacatattctctgagcaacaccctgacactgagcaaggccgactacgagaagcacaaggtgtatgcatgcgaggtgacccaccagggactgagctccccagtgacaaagtccttcaatagaggcgagtgttgataact

SEQ ID NO: 13 - protein, 735 aa, Artificial Sequence
  DMAb13
MDWTWRILFLVAAATGTHAQVQLVESGGGVVQPGGSLRLSCEVSGLTFSNFGMQWVRQAPGKGLEWVAFIRFDGSNKYYADSVKGRFTISRDNSKNTVYLQMGSLRAEDTAVYFCGRVLYGAAADFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCTSSHSLLYSSDNKNYLTWYQQKAGQPPKLLIYWASTRQSGVPDRFSGSGSGTEFTLTISSLQAEDVAVYYCQQYYTKSFTFGQGTKVEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 14 - DNA, 2214 bp, Artificial Sequence
  pGX9261, DMAb13
catggactggacctggagaatcctgttcctggtggcagcagcaaccggaacacacgcacaggtgcagctggtggagagcggcggcggcgtggtgcagcctggcggctctctgagactgagctgcgaggtgtccggcctgaccttcagcaactttggaatgcagtgggtgaggcaggcaccaggcaagggactggagtgggtggccttcatccgctttgacggctctaataagtactatgccgatagcgtgaagggccggttcaccatctctagagacaacagcaagaatacagtgtacctgcagatgggcagcctgagggcagaggacaccgccgtgtacttctgcggacgcgtgctgtatggagcagcagcagatttttggggacagggcaccctggtgacagtgagctccgcctctacaaagggaccaagcgtgtttccactggcaccctctagcaagtccacctctggcggcacagccgccctgggctgtctggtgaaggattacttccccgagcctgtgaccgtgagctggaactccggcgccctgacctccggagtgcacacatttccagccgtgctgcagtcctctggcctgtacagcctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtccagcaccagagctgctgggcggcccaagcgtgttcctgtttccacccaagcctaaggacaccctgatgatctctagaacccccgaggtgacatgcgtggtggtggacgtgagccacgaggaccccgaggtgaagttcaactggtacgtggatggcgtggaggtgcacaatgccaagacaaagcctcgggaggagcagtacaactccacctatagagtggtgtctgtgctgacagtgctgcaccaggactggctgaacggcaaggagtataagtgcaaggtgtccaataaggccctgcctgccccaatcgagaagaccatctctaaggcaaagggacagcctcgggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtattccaagctgaccgtggacaagtctcggtggcagcagggcaacgtgtttagctgctccgtgatgcacgaggccctgcacaatcactacacccagaagtctctgagcctgtccccaggcaagaggggaaggaagcggagatctggcagcggagccacaaacttctccctgctgaagcaggcaggcgatgtggaggagaatccaggacctatggtgctgcagacccaggtgtttatcagcctgctgctgtggatctccggcgcctatggcgacatcgtgatgacacagtccccagattctctggccgtgtccctgggagagagggcaaccatcaactgtacatctagccacagcctgctgtactcctctgacaacaagaattacctgacctggtatcagcagaaggccggccagccacccaagctgctgatctattgggcatccaccaggcagtctggagtgccagaccgcttctccggctctggcagcggcacagagtttaccctgacaatcagctccctgcaggccgaggatgtggccgtgtactattgccagcagtactataccaagagcttcacatttggccagggcaccaaggtggagatcaagacagtggccgcccccagcgtgttcatctttcctccatccgacgagcagctgaagagcggaaccgcatccgtggtgtgcctgctgaacaatttctaccctagggaggccaaggtgcagtggaaggtggataacgccctgcagagcggcaattcccaggagtctgtgaccgagcaggacagcaaggattccacatattccctgtctaacaccctgacactgtccaaggccgattacgagaagcacaaggtgtatgcctgcgaggtgacccaccagggcctgtctagccctgtgacaaagagctttaatcgcggcgagtgttgataact

SEQ ID NO: 15 - protein, 468 aa, Artificial Sequence
  DMAb-34 Heavy Chain
MDWTWRILFLVAAATGTHAQVQLVQSGAEVKKPGASVKVSCKASGYTFSSYGFSWVRQAPGQGLEWMGWISAYNGYTKYAQKFQGRVTMTTDTSTRTAYMELRSLRSDDAAVYYCARDLTPRDGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1431 bp, Artificial Sequence
  DMAb-34 Heavy Chain
ggatccgccgccaccatggactggacatggagaatcctgttcctggtggcagcagcaaccggaacacacgcacaggtgcagctggtgcagtccggagcagaggtgaagaagccaggagcctctgtgaaggtgagctgcaaggcctccggctataccttcagctcctacggcttttcttgggtgcgccaggcacctggacagggcctggagtggatgggctggatcagcgcctacaacggctatacaaagtacgcccagaagttccagggccgggtgaccatgaccacagacaccagcacacgcaccgcctatatggagctgaggagcctgagaagcgacgatgccgccgtgtactattgcgcccgggatctgacccctcgcgacggaatggacgtgtggggacagggaaccacagtgacagtgtctagcgcctctaccaagggcccaagcgtgtttccactggccccctcctctaagtccacatctggcggcaccgccgccctgggatgtctggtgaaggattacttcccagagcccgtgaccgtgtcctggaactctggcgccctgacaagcggcgtgcacacctttccagccgtgctgcagagctccggcctgtattccctgtctagcgtggtgacagtgccctcctctagcctgggcacacagacctacatctgcaacgtgaatcacaagccaagcaataccaaggtggacaagaaggtggagcccaagtcctgtgataagacacacacctgccctccctgtcctgcaccagagctgctgggcggcccatccgtgttcctgtttccacccaagcctaaggacacactgatgatctctaggacacccgaggtgacctgcgtggtggtggacgtgagccacgaggaccccgaggtgaagtttaactggtacgtggatggcgtggaggtgcacaatgccaagaccaagccaagggaggagcagtataattccacatacagagtggtgtctgtgctgaccgtgctgcaccaggactggctgaacggcaaggagtacaagtgcaaggtgtccaataaggccctgcccgcccctatcgagaagacaatctctaaggccaagggccagcctagggagccacaggtgtataccctgcctccatccagagacgagctgacaaagaaccaggtgtctctgacctgtctggtgaagggcttctacccttctgatatcgccgtggagtgggagagcaatggccagccagagaacaattataagaccacaccccctgtgctggacagcgatggctccttctttctgtacagcaagctgacagtggataagtccagatggcagcagggcaacgtgtttagctgttccgtgatgcacgaggccctgcacaatcactacacccagaagtctctgagcctgtcccctggcaagtgataactcgag

SEQ ID NO: 17 - protein, 238 aa, Artificial Sequence
  DMAb-34 Light Chain
MVLQTQVFISLLLWISGAYGDIVLTQSPLSLPVTPGEPASISCRSSQSLLHSNGYNYLDWYLQKPGQPPQVLISLGSNRASGVPDRFSGSGSGTDFTLKISRVEAEDVGVYYCMQALQTPRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 741 bp, Artificial Sequence
  DMAb-34 Light Chain
ggatccgccgccaccatggtgctgcagacccaggtgttcatctccctgctgctgtggatctctggcgcctacggcgacatcgtgctgacccagtctcctctgagcctgcctgtgacaccaggagagccagcaagcatctcctgcaggagctcccagagcctgctgcactccaacggctacaattatctggattggtatctgcagaagccaggacagccccctcaggtgctgatctccctgggctctaacagggcctctggcgtgcctgacagattctctggcagcggctccggaaccgacttcaccctgaagatcagccgggtggaggcagaggacgtgggcgtgtactattgcatgcaggccctgcagacccctcgcacattcggccagggcaccaaggtggagatcaagacagtggccgccccaagcgtgttcatctttccaccctccgacgagcagctgaagtctggcacagccagcgtggtgtgcctgctgaacaacttctacccaagggaggccaaggtgcagtggaaggtggataacgccctgcagagcggcaattcccaggagtctgtgaccgagcaggacagcaaggattccacatattctctgtctagcaccctgacactgtccaaggccgattacgagaagcacaaggtgtatgcatgcgaggtgacccaccagggcctgtcctctcccgtgacaaagagctttaatcggggcgagtgttgataactcgag

SEQ ID NO: 19 - protein, 119 aa, Artificial Sequence
  DMAb-4G7 heavy chain
QVQLQQSGPELEMPGASVKISCKASGSSFTGFSMNWVKQSNGKSLEWIGNIDTYYGGTTYNQKFKGKATLTVDKSSSTAYMQLKSLTSEDSAVYYCARSAYYGSTFAYWGQGTLVTVSS

SEQ ID NO: 20 - DNA, 416 bp, Artificial Sequence
  DMAb-4G7 heavy chain
atggattggacatggaggattctgtttctggtcgccgccgccactggaacccacgcccaggtgcagctgcagcagtcagggcctgagctggaaatgcctggcgcttctgtgaaaatcagttgcaaggcatcaggaagctccttcacagggtttagcatgaactgggtgaaacagagcaatgggaagtccctggagtggatcggcaacattgacacctactatggcggaaccacatacaatcagaagttcaaaggcaaggctacactgactgtggacaaatctagttcaaccgcatatatgcagctgaagagcctgacatccgaggattctgcagtgtactattgcgctagatcagcatactatggcagcactttcgcctactggggccagggaaccctggtcacagtgagctccgc

SEQ ID NO: 21 - protein, 107 aa, Artificial Sequence
  DMAb-4G7 light chain
DIQMTQSPASLSASVGETVTITCRASENIYSYLAWYQQKQGKSPQLLVYNAKTLIEGVPSRFSGSGSGTQFSLKINSLQPEDFGSYFCQHHFGTPFTFGSGTELEIK

SEQ ID NO: 22 - DNA, 639 bp, Artificial Sequence
  DMAb-4G7 light chain
gatattcagatgactcagagccccgcatctctgagtgcctcagtcggcgagacagtgactatcacctgtcgcgcaagtgaaaacatctactcatatctggcctggtaccagcagaaacaggggaagagcccccagctgctggtctataatgctaaaaccctgatcgaaggagtgccttcccgattcagcggcagcgggtctggcacacagtttagcctgaagattaactccctgcagccagaggacttcggcagctacttttgccagcaccatttcggaactcccttcacctttggcagcgggacagagctggaaatcaaaactgtcgcagcccccagtgtgttcatttttccaccctcagacgaacagctgaagtctgggaccgccagtgtcgtgtgtctgctgaacaatttttaccctcgggaggctaaagtccagtggaaggtggataacgcactgcagtctggaaatagtcaggagtcagtgacagaacaggacagcaaagattccacttatagtctgtcaaacacactgactctgtctaaggccgactacgagaaacacaaggtctatgcttgcgaagtgactcatcaggggctgtcctctcctgtgaccaagagcttcaatcgcggcgagtgt

SEQ ID NO: 23 - protein, 141 aa, Artificial Sequence
  DMAb-4 heavy chain
MDWTWRILFLVAAATGTHAQVTLKESGPGILKPSQTLSLTCSLSGFSLSTSGVGVGWFRQPSGKGLEWLALIWWDDDKYYNPSLKSQLSISKDFSRNQVFLKISNVDIADTATYYCARRDPFGYDNAMGYWGQGTSVTVSS

SEQ ID NO: 24 - DNA, 425 bp, Artificial Sequence
  DMAb-4 heavy chain
atggactggacttggagaattctgttcctggtcgcagcagccactgggacacacgcacaggtgacactgaaagagagcggacccggaatcctgaaaccaagccagactctgtccctgacctgcagcctgtccggcttctctctgagtacctcaggagtcggagtgggatggtttcgacagccaagcggaaagggactggagtggctggccctgatctggtgggacgatgacaagtactataacccttcactgaaaagccagctgagcatttccaaggatttctctcgcaaccaggtctttctgaagatcagtaatgtggatattgccgacactgctacctactattgcgctaggagagatccattcggctacgacaatgcaatgggatattggggccagggaacctccgtcacagtgagctccgc

SEQ ID NO: 25 - protein, 107 aa, Artificial Sequence
  DMAb-4 light chain
DIVMTQSQKFMSTSVGDRVSLTCKASQNVGTAVAWYQQKPGQSPKLLIYSASNRYTGVPDRFTGSGSGTDFTLTISNMQSEDLADYFCQQYSSYPLTFGAGTKLEIK

SEQ ID NO: 26 - DNA, 639 bp, Artificial Sequence
  DMAb-4 light chain
gatattgtgatgacccagagccagaagttcatgtccacatctgtcggcgaccgggtgtctctgacctgtaaggccagtcagaacgtcggaactgctgtggcatggtatcagcagaagcctgggcagtccccaaaactgctgatctacagtgcttcaaacagatataccggcgtgcctgatcggttcaccggaagcgggtccggcacagactttactctgaccatttctaatatgcagagtgaagatctggctgactacttctgccagcagtactcctcttatccactgacatttggagcagggactaagctggaaatcaaaacagtcgcagccccctccgtgttcatttttccaccctctgatgagcagctgaagtcaggcactgccagcgtcgtgtgtctgctgaacaatttctaccccagggaggccaaggtccagtggaaagtggacaacgctctgcagagcggaaattcccaggagtctgtgactgaacaggatagtaaagactcaacctattctctgagtaacacactgactctgtccaaggcagactacgagaagcacaaagtctatgcctgcgaagtgacccatcagggcctgagttcaccagtgacaaagtcttttaatcgcggagagtgt

(19) United States
(12) Patent Application Publication
(10) Pub. No.: US 2019/0290750 A1
(43) Pub. Date: Sep. 26, 2019

(54) DNA ANTIBODY CONSTRUCTS FOR USE AGAINST EBOLA VIRUS

(71) Applicants: The Trustees of the University of Pennsylvania, Philadelphia, PA (US); THE WISTARE INSTITUTE OF ANATOMY AND BIOLOGY, Philadelphia, PA (US)

(72) Inventors: David WEINER, Merion, PA (US); Ami PATEL, Philadelphia, PA (US)

(21) Appl. No.: 16/465,680

(22) PCT Filed: Dec. 1, 2017
(86) PCT No.: PCT/US2017/064161

§ 371 Date: May 31, 2019

Related U.S. Application Data
(60) Provisional application No. 62/429,454, filed on Dec. 2, 2016.
(60) Provisional application No. 62/504,436, filed on May 10, 2017.
(60) Provisional application No. 62/559,422, filed on Sep. 15, 2017.

(51) Int. Cl. A61K 39/12; A61K 35/76; A61K 39/42; C07K 16/10; A61P 31/14; C07K 14/18

(52) U.S. Cl. CPC A61K 39/12; A61K 35/76; A61K 39/42; A61K 2039/505; A61P 31/14; C07K 14/1825; C07K 16/10

(57) ABSTRACT

Disclosed herein is a composition including a recombinant nucleic acid sequence that encodes an antibody to an Ebola viral antigen. Also disclosed herein is a method of generating a synthetic antibody in a subject by administering the composition to the subject. The disclosure also provides a method of preventing and/or treating an Ebola virus infection in a subject using said composition and method of generation.

### CROSS-REFERENCE TO RELATED APPLICATIONS

The present application claims priority to U.S. Provisional Application No. 62/429,454, filed Dec. 2, 2016, U.S. Provisional Application No. 62/504,436, filed May 10, 2017, and U.S. Provisional Application No. 62/559,422, filed Sep. 15, 2017, each of which is incorporated by reference herein in its entirety.

### STATEMENT OF GOVERNMENT INTEREST

This invention was made with government support under W31P4Q-15-1-0003 awarded by Defense Advanced Research Projects Agency (DARPA). The government has certain rights in the invention.

### TECHNICAL FIELD

The present invention relates to a composition comprising a recombinant nucleic acid sequence for generating one or more synthetic antibodies, and functional fragments thereof, in vivo, and a method of preventing and/or treating viral infection in a subject by administering said composition.

### BACKGROUND

Monoclonal antibodies (mAbs) targeting the Ebola virus glycoprotein (GP) represent an important treatment approach against Ebola virus disease (EVD). It has been shown that individual mAbs and mAb cocktails can successfully protect small animals and non-human primates against lethal Ebola virus infection. MAb-based therapy against EVD is further supported by favorable recovery in confirmed human EVD cases that received the anti-GP mAb cocktail, ZMapp. However, the dramatic cost, slow development, and requirement for several high-dose administrations (mg/kg) represent a significant challenge for protein mAb delivery, especially during a possible outbreak.

Thus, there is need in the art for improved therapeutics that prevent and/or treat Ebola virus infection. The current invention satisfies this need.

### SUMMARY

The present invention is directed to a nucleic acid molecule encoding one or more synthetic antibodies, wherein the nucleic acid molecule comprises at least one selected from the group consisting of a) a nucleotide sequence encoding an anti-Ebola virus glycoprotein (GP) synthetic antibody; and b) a nucleotide sequence encoding a fragment of an anti-Ebola virus glycoprotein (GP) synthetic antibody.

In one embodiment, the nucleic acid molecule comprises a nucleotide sequence encoding a cleavage domain.

In one embodiment, the nucleic acid molecule comprises a nucleotide sequence encoding one or more of a variable heavy chain region and a variable light chain region of an anti-Ebola virus GP antibody.

In one embodiment, the nucleic acid molecule encodes one or more synthetic bispecific antibodies. A bispecific antibody molecule according to the invention may have two binding sites of any desired specificity. In some embodiments one of the binding sites is capable of binding a Ebola virus antigen. In some embodiment, one of the binding sites is capable of binding a cell surface marker on an immune cell.

In one embodiment, the nucleic acid molecule comprises a nucleotide sequence encoding one or more sequences as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, and SEQ ID NO:25.

In one embodiment, the nucleic acid molecule comprises a nucleotide sequence encoding a variable heavy chain region and a variable light chain region of an anti-Ebola virus GP antibody. In one embodiment, the nucleic acid molecule comprises a nucleotide sequence comprising a sequence selected from the group comprising SEQ ID NO:2, SEQ ID NO:4, SEQ ID NO:6, SEQ ID NO:8, SEQ ID NO:10, SEQ ID NO:12, SEQ ID NO:14 SEQ ID NO: 16, SEQ ID NO:18, SEQ ID NO:20, SEQ ID NO:22, SEQ ID NO:24, and SEQ ID NO:26.

In one embodiment, the nucleic acid molecule comprises a nucleotide sequence encoding a polypeptide comprising a variable heavy chain region; an IRES element; and a variable light chain region. In one embodiment, the IRES element is one of a viral IRES or an eukaryotic IRES.

In one embodiment, the nucleic acid molecule comprises a nucleotide sequence having at least about 95% identity over an entire length of the nucleic acid sequence to a nucleic acid encoding an sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, and SEQ ID NO:25.

In one embodiment, the nucleic acid molecule comprises a nucleotide sequence encoding a sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, and SEQ ID NO:25.

In one embodiment, the nucleic acid molecule comprises a nucleotide sequence having at least about 95% identity over an entire length of the nucleic acid sequence to a nucleic acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO: 16, SEQ ID NO:18, SEQ ID NO:20, SEQ ID NO:22, SEQ ID NO:24, and SEQ ID NO:26.

In one embodiment, the nucleic acid molecule comprises a nucleotide sequence selected from the group consisting of SEQ ID NO:2, SEQ ID NO:4, SEQ ID NO:6, SEQ ID NO:8, SEQ ID NO:10, SEQ ID NO:12, SEQ ID NO:14 SEQ ID NO: 16, SEQ ID NO:18, SEQ ID NO:20, SEQ ID NO:22, SEQ ID NO:24, and SEQ ID NO:26.

In one embodiment, the nucleic acid molecule comprises a RNA sequence transcribed from a DNA sequence described herein. For example, in one embodiment, the nucleic acid molecule comprises a RNA sequence transcribed by the DNA sequence of SEQ ID NOs:

SEQ ID NO:1, SEQ ID NO:3, SEQ ID NO:5, SEQ ID NO:7, SEQ ID NO:9, SEQ ID NO:11, SEQ ID NO:13, SEQ ID NO: 15, or SEQ ID NO:17, or a variant thereof or a fragment thereof. In another embodiment, the nucleic acid molecule comprises an RNA sequence transcribed by a DNA sequence encoding the polypeptide sequence of SEQ ID NOs: SEQ ID NO:2, SEQ ID NO:4, SEQ ID NO:6, SEQ ID NO:8, SEQ ID NO:10, SEQ ID NO:12, SEQ ID NO:14 SEQ ID NO: 16, or SEQ ID NO:18, or a variant thereof or a fragment thereof

In one embodiment, the nucleotide sequence encodes a leader sequence. In one embodiment, the nucleic acid molecule comprises an expression vector.

The invention further provides a composition comprising any of the nucleic acid molecules described herein.

In one embodiment, the composition comprises a pharmaceutically acceptable excipient.

The invention further relates to a method of preventing or treating a disease in a subject, the method comprising administering to the subject a nucleic acid molecule or a composition as described herein.

In one embodiment, the disease is an Ebola virus infection.

In one embodiment, the method further comprises administering an antibiotic agent to the subject. In one embodiment, an antibiotic is administered less than 10 days after administration of the nucleic acid molecule or composition.

In one embodiment, the method further comprises administering an antibiotic agent to the subject. In one embodiment an antibiotic is administered less than 10 days after administration of the nucleic acid molecule or composition.

In one embodiment, the invention provides novel sequences for use for producing monoclonal antibodies in mammalian cells or for delivery in DNA or RNA vectors including bacterial, yeast, as well as viral vectors.

### BRIEF DESCRIPTION OF THE DRAWINGS

FIG. 1, comprising FIG. 1A and FIG. 1B, depicts a schematic of Protein MAb formulation and delivery (FIG. 1A) and DMAb design, formulation and delivery (FIG. 1B)

FIG. 2 is a set of immunofluorescence images depicting DMAb expression in muscle.

FIG. 3, comprising FIG. 3A and FIG. 3B, depicts the results of experiments demonstrating in vitro expression of EVD DMAbs. FIG. 3A depicts the structure of GP.

FIG. 3B depicts a Western blot demonstrating the expression of DMAb heavy and light chains in HEK239T cells transfected with DMAb.

FIG. 4, comprising FIG. 4A and FIG. 4B, depicts the results of experiments demonstrating the in vivo expression of EVD DMAbs. BALB/c mice received 400 ug of EVD DMAb by intramuscular injection, followed by electroporation. Peak expression levels of human IgG1 in mouse serum are displayed in the graph (FIG. 4A). EVD DMAb circulating levels can be further enhanced through optimizing sequence, expression, and delivery (FIG. 4B).

FIG. 5 depicts a schematic of employing DNA vectors for delivery of mAb (DMAb) against infectious diseases.

FIG. 6 depicts a schematic of EVD DMAbs.

FIG. 7 depicts experimental results demonstrating that in vivo expressed EVD DMAb-11 is comparable to protein IgG.

FIG. 8 depicts experimental results demonstrating that EVD DMAbs provides preventative protection against lethal mouse-adapted EBOV challenge.

FIG. 9 depicts experimental results demonstrating that EVD DMAbs offer long-lasting preventative protection.

FIG. 10 depicts experimental results demonstrating that multiple EVD DMAbs can be administered at the same time.

FIG. 11 depicts a summary of the protective effects of EVD DMAbs.

FIG. 12, comprising FIG. 12A through FIG. 12C, depicts experimental results demonstrating DMAb in vivo protection from EVD. FIG. 12A depicts the experimental design. BALB/c mice received EVD DMAb on Day-28 before challenge, by intramuscular injection, followed by electroporation. FIG. 12B depicts expression levels of human IgG1 in mouse serum on day 14 following DMAb injection. FIG. 12C depicts survival following lethal challenge with 1000LD₅₀ of mouse-adapted Ebola virus (Mayinga).

FIG. 13 depicts experimental results demonstrating Cmax expression levels in BALB/c mice (μg/mL).

FIG. 14 depicts experimental results demonstrating the in vivo expression and characterization of DMAb-11 and DMAb-34.

FIG. 15 depicts experimental results demonstrating EVD DMAb-11 epitope mapping.

FIG. 16 depicts experimental results demonstrating that EVD DMAb-11 and EVD-34 protect against lethal mouse-adapted Mayinga challenge.

FIG. 17 depicts a schematic diagram of DMAb platform. DMAbs encode anti-GP mAb Ig heavy and light chains as a single polycistronic construct with a cleavage site or as two independent transgenes. DMAbs are delivered directly in vivo where they are expressed, undergo folding and assembly, and are secreted directly into systemic circulation.

FIG. 18, comprising FIG. 18A and FIG. 18B, depicts EVD DMAb Engineering and optimization. FIG. 18A depicts evaluation of GP-DMAbs DMAb-5, DMAb-7, DMAb-11 and DMAb-34. FIG. 18B depicts experimental results demonstrating variations of these optimizations were performed for 27 different anti-EVD DMAbs

FIG. 19, comprising FIG. 19A and FIG. 19B depicts experimental results demonstrating DMAb-11 and DMAb-34 expression. FIG. 19A depicts results demonstrating DMAb-11 and DMAb-34 in vitro expression. FIG. 19B depicts results demonstrating DMAb-11 and DMAb-34 in vivo expression.

FIG. 20, comprising FIG. 20A through FIG. 20F, depicts experimental results demonstrating expression and characterization of DMAb-11 and DMAb-34. FIG. 20A depicts experimental results demonstrating the long-term expression of different doses of DMAb-11 monitored in parallel with single injection of different doses of protein 5.6.1A2.

FIG. 20B depicts experimental results demonstrating the long-term expression of different doses of DMAb-34 monitored in parallel with single injection of different doses of protein 15784. FIG. 20C depicts DMAb-11 and protein mAb11 binding to 1976-GP. FIG. 20D depicts DMAb-34 protein mAb34 binding to 1976-GP. FIG. 20E depicts DMAb-11 neutralization of live EBOV-GFP (strain Mayinga) virus in a neutralization assay. FIG. 20F depicts DMAb-34 neutralization of live EBOV-GFP (strain Mayinga) virus in a neutralization assay.

FIG. 21, comprising FIG. 21A and FIG. 21B, depicts epitope mapping by alanine scanning of EBOV GP using HEK 293T cells. FIG. 21A depicts epitope mapping of DMAb-1A2. FIG. 21B depicts epitope mapping of DMAb-784.

FIG. 22, comprising FIG. 22A through FIG. 22D, depicts experimental results demonstrating EVD DMAbs are protective in vivo. FIG. 22A depicts an overview of injection regimen. DMAbs were administered 28 days before lethal challenge. Animals were monitored for 21 days post-challenge administration. FIG. 22B depicts protection and % weight change in animals receiving a negative and positive control. FIG. 22C depicts protection, % weight change, and DMAb expression in animals receiving DMAb-11 (n=30).

FIG. 22D depicts protection, % weight change, and DMAb expression in animals receiving DMAb-34. (n=10).

FIG. 23 depicts experimental results demonstrating the codelivery of DMAb-11 and DMAb-34 at separate injections sites on the mouse leg.

FIG. 24 depicts experimental results demonstrating that DMAb co-formulation expresses at comparable in vivo levels to pre-treatment.

FIG. 25, depicts experimental results demonstrating DMAb-11 expression at different and protection, and % weight change in animals receiving of DMAb-11.

FIG. 26, comprising FIG. 26A through FIG. 26D, depicts experimental results demonstrating that EVD DMAbs are partially protection 3 months following administration. FIG. 26A depicts an overview of injection regimen. DMAbs were administered 82 days before lethal challenge. Animals were monitored for 21 days post-challenge administration. FIG. 26B depicts DMAb-11 expression at day −26 before challenge (day 56 post-DMAb administration) (n=10). FIG. 26C depicts protection in animals receiving DMAb-11. FIG. 26D depicts % weight change (n=10).

### DETAILED DESCRIPTION

The present invention relates to compositions comprising a recombinant nucleic acid sequence encoding an antibody, a fragment thereof, a variant thereof, or a combination thereof. The composition can be administered to a subject in need thereof to facilitate in vivo expression and formation of a synthetic antibody.

In particular, the heavy chain and light chain polypeptides expressed from the recombinant nucleic acid sequences can assemble into the synthetic antibody. The heavy chain polypeptide and the light chain polypeptide can interact with one another such that assembly results in the synthetic antibody being capable of binding the antigen, being more immunogenic as compared to an antibody not assembled as described herein, and being capable of eliciting or inducing an immune response against the antigen.

Additionally, these synthetic antibodies are generated more rapidly in the subject than antibodies that are produced in response to antigen induced immune response. The synthetic antibodies are able to effectively bind and neutralize a range of antigens. The synthetic antibodies are also able to effectively protect against and/or promote survival of disease.

A sequence listing provided herewith contains a list of 14 sequences including the following:

SEQ ID NO:1 is the amino acid sequence of DMAb-2G4.

SEQ ID NO:2 is the nucleotide sequence of DMAb-2G4, pGX9226.

SEQ ID NO:3 is the amino acid sequence of DMAb-4G7.

SEQ ID NO:4 is the nucleotide sequence of DMAb-4G7, pGX9229.

SEQ ID NO:5 is the amino acid sequence of DMAb-4.

SEQ ID NO:6 is the nucleotide sequence of DMAb-4, pGX9230.

SEQ ID NO:7 is the amino acid sequence of DMAb-10.

SEQ ID NO:8 is the nucleotide sequence of DMAb-10, pGX9244.

SEQ ID NO:9 is the amino acid sequence of DMAb-11.

SEQ ID NO:10 is the nucleotide sequence of DMAb-11, pGX9256.

SEQ ID NO:11 is the amino acid sequence of DMAb-12.

SEQ ID NO:12 is the nucleotide sequence of DMAb-12, pGX9260.

SEQ ID NO:13 is the amino acid sequence of DMAb-13.

SEQ ID NO:14 is the nucleotide sequence of DMAb-13, pGX9261.

SEQ ID NO:15 is the amino acid sequence of DMAb-34 heavy chain.

SEQ ID NO:16 is the nucleotide sequence of DMAb-34 heavy chain.

SEQ ID NO:17 is the amino acid sequence of DMAb-34 light chain.

SEQ ID NO:18 is the nucleotide sequence of DMAb-34 light chain.

SEQ ID NO:19 is the amino acid sequence of DMAb-4G7 heavy chain.

SEQ ID NO:20 is the nucleotide sequence of DMAb-4G7 heavy chain.

SEQ ID NO:21 is the amino acid sequence of DMAb-4G7 light chain.

SEQ ID NO:22 is the nucleotide sequence of DMAb-4G7 light chain.

SEQ ID NO:23 is the amino acid sequence of DMAb-4 heavy chain.

SEQ ID NO:24 is the nucleotide sequence of DMAb-4 heavy chain.

SEQ ID NO:25 is the amino acid sequence of DMAb-4 light chain.

SEQ ID NO:26 is the nucleotide sequence of DMAb-4 light chain.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITION

The present invention relates to a composition comprising a recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

In one embodiment, the composition comprises a nucleotide sequence encoding a synthetic antibody. In one embodiment, the composition comprises a nucleic acid molecule comprising a first nucleotide sequence encoding a first synthetic antibody and a second nucleotide sequence encoding a second synthetic antibody. In one embodiment, the nucleic acid molecule comprises a nucleotide sequence encoding a cleavage domain.

In one embodiment, the nucleic acid molecule comprises a nucleotide sequence encoding an anti-Ebola Glycoprotein (anti-Ebola GP) antibody.

In one embodiment, the nucleotide sequence encoding an anti-Ebola GP antibody comprises one or more codon optimized nucleic acid sequences encoding an amino acid sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, or SEQ ID NO:25.

In one embodiment, the nucleotide sequence encoding an anti-Ebola GP antibody comprises one or more RNA sequence transcribed from one or more DNA sequences encoding an amino acid sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, and SEQ ID NO:25.

In one embodiment, the nucleotide sequence encoding an anti-Ebola GP antibody comprises one or more codon optimized nucleic acid sequences as set forth in SEQ ID NO:2, SEQ ID NO:4, SEQ ID NO:6, SEQ ID NO:8, SEQ ID NO:10, SEQ ID NO:12, SEQ ID NO:14 SEQ ID NO: 16, SEQ ID NO:18, SEQ ID NO:20, SEQ ID NO:22, SEQ ID NO:24, or SEQ ID NO:26.

In one embodiment, the nucleotide sequence encoding an anti-Ebola GP antibody comprises one or more RNA sequence transcribed from one or more DNA sequences as set forth in SEQ ID NO:2, SEQ ID NO:4, SEQ ID NO:6, SEQ ID NO:8, SEQ ID NO:10, SEQ ID NO:12, SEQ ID NO:14 SEQ ID NO: 16, SEQ ID NO:18, SEQ ID NO:20, SEQ ID NO:22, SEQ ID NO:24, or SEQ ID NO:26.

The composition of the invention can treat, prevent and/or protect against any disease, disorder, or condition associated with Ebola infection. In certain embodiments, the composition can treat, prevent, and or/protect against viral infection. In certain embodiments, the composition can treat, prevent, and or/protect against condition associated with Ebola infection.

The composition can result in the generation of the synthetic antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody in the subject within at least about 1 day, 2 days, 3 days, 4 days, 5 days, 6 days, 7 days, 8 days, 9 days, or 10 days of administration of the composition to the subject. The composition can result in generation of the synthetic antibody in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody in the subject more quickly than the generation of an endogenous antibody in a subject who is administered an antigen to induce a humoral immune response. The composition can result in the generation of the synthetic antibody at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

### 3. RECOMBINANT NUCLEIC ACID SEQUENCE

As described above, the composition can comprise a recombinant nucleic acid sequence. The recombinant nucleic acid sequence can encode the antibody, a fragment thereof, a variant thereof, or a combination thereof. The antibody is described in more detail below.

The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; addition of an internal IRES sequence and eliminating to the extent possible cis-acting sequence motifs (i.e., internal TATA boxes).

### Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

(1) Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

(2) Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

(3) Protease Cleavage Site

The recombinant nucleic acid sequence construct can include heterologous nucleic acid sequence encoding a protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

(4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

(5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

(6) Intron

The recombinant nucleic acid sequence construct can include one or more introns. Each intron can include functional splice donor and acceptor sites. The intron can include an enhancer of splicing. The intron can include one or more signals required for efficient splicing.

(7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

(8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

(9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

(10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human (3-globin polyadenylation signal. The SV40 polyadenylation signal may be a polyadenylation signal from a pCEP4 plasmid (Invitrogen, San Diego, Calif.).

(11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

### Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

(12) Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide. For example, in one embodiment, the first recombinant nucleic acid sequence encodes a heavy chain polypeptide having an amino acid sequence at least 95% homologous to SEQ ID NO: 15. In one embodiment, the first recombinant nucleic acid sequence comprises a nucleic acid sequence at least 95% homologous to SEQ ID NO: 16. In one embodiment, the second recombinant nucleic acid sequence encodes a heavy chain polypeptide having an amino acid sequence at least 95% homologous to SEQ ID NO: 17. In one embodiment, the first recombinant nucleic acid sequence comprises a nucleic acid sequence at least 95% homologous to SEQ ID NO: 18.

The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

Accordingly, one example of arrangement 1 can include the first vector (and thus first recombinant nucleic acid sequence construct) encoding the heavy chain polypeptide that includes VH and CH1, and the second vector (and thus second recombinant nucleic acid sequence construct) encoding the light chain polypeptide that includes VL and CL. A second example of arrangement 1 can include the first vector (and thus first recombinant nucleic acid sequence construct) encoding the heavy chain polypeptide that includes VH, CH1, hinge region, CH2, and CH3, and the second vector (and thus second recombinant nucleic acid sequence construct) encoding the light chain polypeptide that includes VL and CL.

(13) Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include the heterologous nucleic acid sequence encoding the protease cleavage site and/or the linker sequence. If included in the recombinant nucleic acid sequence construct, the heterologous nucleic acid sequence encoding the protease cleavage site can be positioned between the heterologous nucleic acid sequence encoding the heavy chain polypeptide and the heterologous nucleic acid sequence encoding the light chain polypeptide. Accordingly, the protease cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Accordingly, one example of arrangement 2 can include the vector (and thus recombinant nucleic acid sequence construct) encoding the heavy chain polypeptide that includes VH and CH1, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A second example of arrangement of 2 can include the vector (and thus recombinant nucleic acid sequence construct) encoding the heavy chain polypeptide that includes VH and CH1, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

A third example of arrangement 2 can include the vector (and thus recombinant nucleic acid sequence construct) encoding the heavy chain polypeptide that includes VH, CH1, hinge region, CH2, and CH3, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A forth example of arrangement of 2 can include the vector (and thus recombinant nucleic acid sequence construct) encoding the heavy chain polypeptide that includes VH, CH1, hinge region, CH2, and CH3, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

(14) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

(15) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, Calif.), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

(16) RNA

In one embodiment, the nucleic acid is an RNA molecule. In one embodiment, the RNA molecule is transcribed from a DNA sequence described herein. For example, in some embodiments, the RNA molecule is encoded by a DNA sequence at least 90% homologous to one of SEQ ID NOs: 2, 4, 6, 8, 10, 12, 14, 16, 18, 20, 22, 24, or 26. In another embodiment, the nucleotide sequence comprises an RNA sequence transcribed by a DNA sequence encoding a polypeptide sequence of SEQ ID NOs:1, 3, 5, 7, 9, 11, 13, 15, 17, 19, 21, 23, or 25, or a variant thereof or a fragment thereof. Accordingly, in one embodiment, the invention provides an RNA molecule encoding one or more of the DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

(17) Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be perM2. The LEC can be perNP. perNP and perMR can be derived from pNP (Puerto Rico/34) and pM2 (New Caledonia/99), respectively.

(18) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

(19) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 4. ANTIBODY

As described above, the recombinant nucleic acid sequence can encode the antibody, a fragment thereof, a variant thereof, or a combination thereof. The antibody can bind or react with the antigen, which is described in more detail below.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

In one embodiment, the antibody binds an ebolavirus antigen. In one embodiment, the antibody binds an ebolavirus glycoprotein. In one embodiment, the antibody binds at least one an epitope of an ebolavirus glycoprotein. For example, in one embodiment, the antibody binds ebolavirus glycoprotein epitope comprising the reside W531, I527, or both.

The antibody may be modified to reduce or prevent antibody-dependent enhancement (ADE) of disease associated with the antigen as described in more detail below.

### Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments one of the binding sites is capable of binding a tumor associated antigen. In some embodiments, the binding site included in the Fab fragment is a binding site specific for a Ebolavirus antigen. In some embodiments, the binding site included in the single chain Fv fragment is a binding site specific for a Ebolavirus antigen such as a Ebolavirus glycoprotein antigen.

In some embodiments, one of the binding sites of an antibody molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing .alpha.- and .beta.-chains, in some embodiments it encompasses .gamma.-chains and .delta.-chains (supra). Accordingly, in some embodiments the TCR is TCR (alpha/beta) and in some embodiments it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the antibody molecule binds a Ebolavirus antigen and the second binding site binds a T cell specific receptor molecule and/or a natural killer (NK) cell specific receptor molecule.

In some embodiments, the first binding site of the antibody molecule binds one of Ebolavirus GP glycan cap, Ebolavirus GP fusion loop, or Ebolavirus GP chalice base, and the second binding site binds a T cell specific receptor molecule and/or a natural killer (NK) cell specific receptor molecule. In some embodiments, the first binding site of the antibody molecule binds a Ebolavirus antigen and the second binding site binds one of CD3, the T cell receptor (TCR), CD28, CD16, NKG2D, Ox40, 4-1BB, CD2, CD5 and CD95.

In some embodiments, the first binding site of the antibody molecule binds a T cell specific receptor molecule and/or a natural killer (NK) cell specific receptor molecule and the second binding site binds an Ebolavirus antigen. In some embodiments, the first binding site of the antibody binds a T cell specific receptor molecule and/or a natural killer (NK) cell specific receptor molecule and the second binding site binds one of Ebolavirus GP glycan cap, Ebolavirus GP fusion loop, or Ebolavirus GP chalice base. In some embodiments, the first binding site of the antibody binds one of CD3, the T cell receptor (TCR), CD28, CD16, NKG2D, Ox40, 4-1BB, CD2, CD5 and CD95, and the second binding site binds an Ebolavirus antigen.

### Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

### Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, 0-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### Reduced ADE Response

The antibody may be modified to reduce or prevent antibody-dependent enhancement (ADE) of disease associated with the antigen, but still neutralize the antigen.

In some embodiments, the antibody may be modified to include one or more amino acid substitutions that reduce or prevent binding of the antibody to FcγR1a. The one or more amino acid substitutions may be in the constant region of the antibody. The one or more amino acid substitutions may include replacing a leucine residue with an alanine residue in the constant region of the antibody, i.e., also known herein as LA, LA mutation or LA substitution. The one or more amino acid substitutions may include replacing two leucine residues, each with an alanine residue, in the constant region of the antibody and also known herein as LALA, LALA mutation, or LALA substitution. The presence of the LALA substitutions may prevent or block the antibody from binding to FcγR1a, and thus, the modified antibody does not enhance or cause ADE of disease associated with the antigen, but still neutralizes the antigen.

### 5. ANTIGEN

The synthetic antibody is directed to the antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be from a virus. The antigen can be associated with viral infection. In one embodiment, the antigen can be associated with Ebola infection. In one embodiment, the antigen can be an Ebola glycoprotein.

In one embodiment, the antigen can be a fragment of an Ebola glycoprotein. For example, in one embodiment, the antigen is a fragment of an Ebola glycoprotein, wherein the fragment comprises the amino acid Trp531. In one embodiment, the antigen is a fragment of an Ebola glycoprotein, wherein the fragment comprises the amino acid Ile527. In one embodiment, the antigen is a fragment of an Ebola glycoprotein, wherein the fragment comprises the amino acids Trp531 and Ile527.

In one embodiment, a synthetic antibody of the invention targets two or more antigens. In one embodiment, at least one antigen of a bispecific antibody is selected from the antigens described herein. In one embodiment, the two or more antigens are selected from the antigens described herein.

### Viral Antigens

The viral antigen can be a viral antigen or fragment or variant thereof. The virus can be a disease causing virus. The virus can be the Ebola virus.

The antigen may be a Ebola viral antigen, or fragment thereof, or variant thereof. The Ebola antigen can be from a factor that allows the virus to replicate, infect or survive. Factors that allow a Ebola virus to replicate or survive include, but are not limited to structural proteins and non-structural proteins. Such a protein can be an envelope protein or a glycoprotein.

In one embodiment, an envelope protein is Ebola GP.

### 6. EXCIPIENTS AND OTHER COMPONENTS OF THE COMPOSITION

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### 7. METHOD OF GENERATING THE SYNTHETIC ANTIBODY

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 8. METHOD OF IDENTIFYING OR SCREENING FOR THE ANTIBODY

The present invention further relates to a method of identifying or screening for the antibody described above, which is reactive to or binds the antigen described above. The method of identifying or screening for the antibody can use the antigen in methodologies known in those skilled in art to identify or screen for the antibody. Such methodologies can include, but are not limited to, selection of the antibody from a library (e.g., phage display) and immunization of an animal followed by isolation and/or purification of the antibody.

### 9. METHOD OF DELIVERY OF THE COMPOSITION

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, Pa.) or Elgen electroporator (Inovio Pharmaceuticals, Plymouth Meeting, Pa.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### 10. METHOD OF TREATMENT

Also provided herein is a method of treating, protecting against, and/or preventing disease in a subject in need thereof by generating the synthetic antibody in the subject. The method can include administering the composition to the subject. Administration of the composition to the subject can be done using the method of delivery described above.

In certain embodiments, the invention provides a method of treating protecting against, and/or preventing a Ebola Virus infection. In one embodiment, the method treats, protects against, and/or prevents a disease associated with Ebola Virus.

Upon generation of the synthetic antibody in the subject, the synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The synthetic antibody can treat, prevent, and/or protect against disease in the subject administered the composition. The synthetic antibody by binding the antigen can treat, prevent, and/or protect against disease in the subject administered the composition. The synthetic antibody can promote survival of the disease in the subject administered the composition. In one embodiment, the synthetic antibody can provide increased survival of the disease in the subject over the expected survival of a subject having the disease who has not been administered the synthetic antibody. In various embodiments, the synthetic antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition. In one embodiment, the synthetic antibody can provide increased protection against the disease in the subject over the expected protection of a subject who has not been administered the synthetic antibody. In various embodiments, the synthetic antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### 11. USE IN COMBINATION WITH ANTIBIOTICS

The present invention also provides a method of treating, protecting against, and/or preventing disease in a subject in need thereof by administering a combination of the synthetic antibody and a therapeutic antibiotic agent.

The synthetic antibody and an antibiotic agent may be administered using any suitable method such that a combination of the synthetic antibody and antibiotic agent are both present in the subject. In one embodiment, the method may comprise administration of a first composition comprising a synthetic antibody of the invention by any of the methods described in detail above and administration of a second composition comprising an antibiotic agent less than 1, less than 2, less than 3, less than 4, less than 5, less than 6, less than 7, less than 8, less than 9 or less than 10 days following administration of the synthetic antibody. In one embodiment, the method may comprise administration of a first composition comprising a synthetic antibody of the invention by any of the methods described in detail above and administration of a second composition comprising an antibiotic agent more than 1, more than 2, more than 3, more than 4, more than 5, more than 6, more than 7, more than 8, more than 9 or more than 10 days following administration of the synthetic antibody. In one embodiment, the method may comprise administration of a first composition comprising an antibiotic agent and administration of a second composition comprising a synthetic antibody of the invention by any of the methods described in detail above less than 1, less than 2, less than 3, less than 4, less than 5, less than 6, less than 7, less than 8, less than 9 or less than 10 days following administration of the antibiotic agent. In one embodiment, the method may comprise administration of a first composition comprising an antibiotic agent and administration of a second composition comprising a synthetic antibody of the invention by any of the methods described in detail above more than 1, more than 2, more than 3, more than 4, more than 5, more than 6, more than 7, more than 8, more than 9 or more than 10 days following administration of the antibiotic agent. In one embodiment, the method may comprise administration of a first composition comprising a synthetic antibody of the invention by any of the methods described in detail above and a second composition comprising an antibiotic agent concurrently. In one embodiment, the method may comprise administration of a first composition comprising a synthetic antibody of the invention by any of the methods described in detail above and a second composition comprising an antibiotic agent concurrently. In one embodiment, the method may comprise administration of a single composition comprising a synthetic antibody of the invention and an antibiotic agent.

Non-limiting examples of antibiotics that can be used in combination with the synthetic antibody of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

The present invention has multiple aspects, illustrated by the following non-limiting examples.

### 12. GENERATION OF SYNTHETIC ANTIBODIES IN VITRO AND EX VIVO

In one embodiment, the synthetic antibody is generated in vitro or ex vivo. For example, in one embodiment, a nucleic acid encoding a synthetic antibody can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### 13. EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

The studies presented herein demonstrate the generation of functional anti-Ebola “DNA monoclonal antibodies” (DMAb) via intramuscular electroporation of plasmid DNA.

As described herein, an optimized, synthetic DNA vector platform (DMAb) to deliver encoded mAb heavy and light chains directly into skeletal muscle was designed, employing the cells as biological factories that will secrete a functional antibody at detectable levels in systemic circulation. DMAbs encoding anti-GP mAbs that target the GP glycan cap, fusion loop, and chalice base were developed. BALB/c mice were administered individual DMAbs or a combination of multiple DMAbs by intramuscular (IM) DNA injection, followed by in vivo electroporation (EP). EVD DMAbs were detected in mouse serum for >3 months, with Cmax serum levels comparable to protein-delivered human IgG1. In the field, EVD DMAbs could be administered in advance during a possible EVD outbreak.

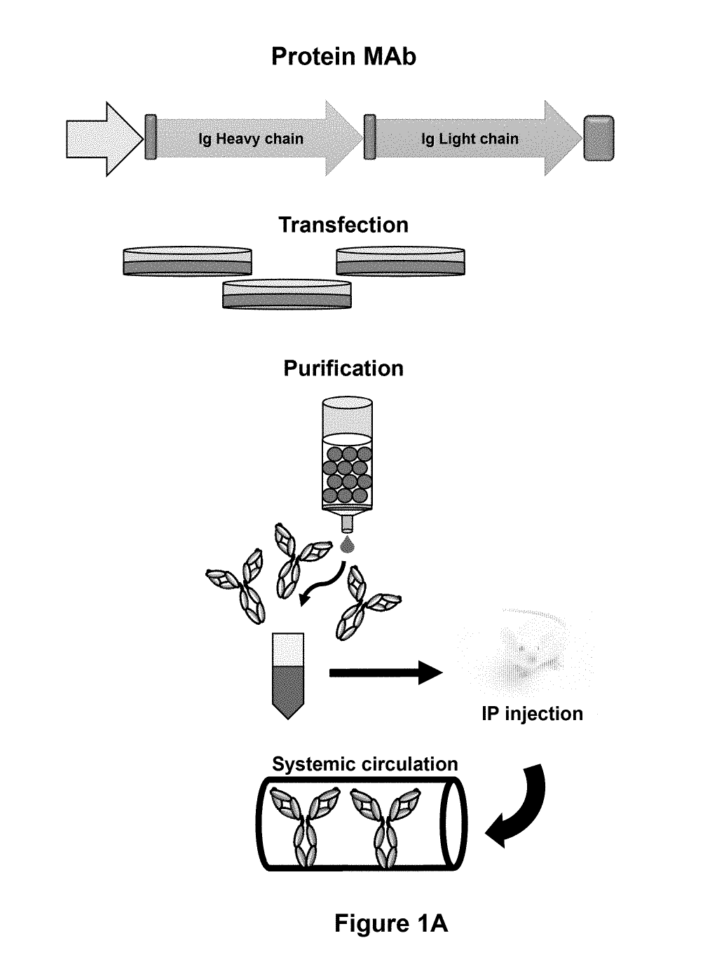

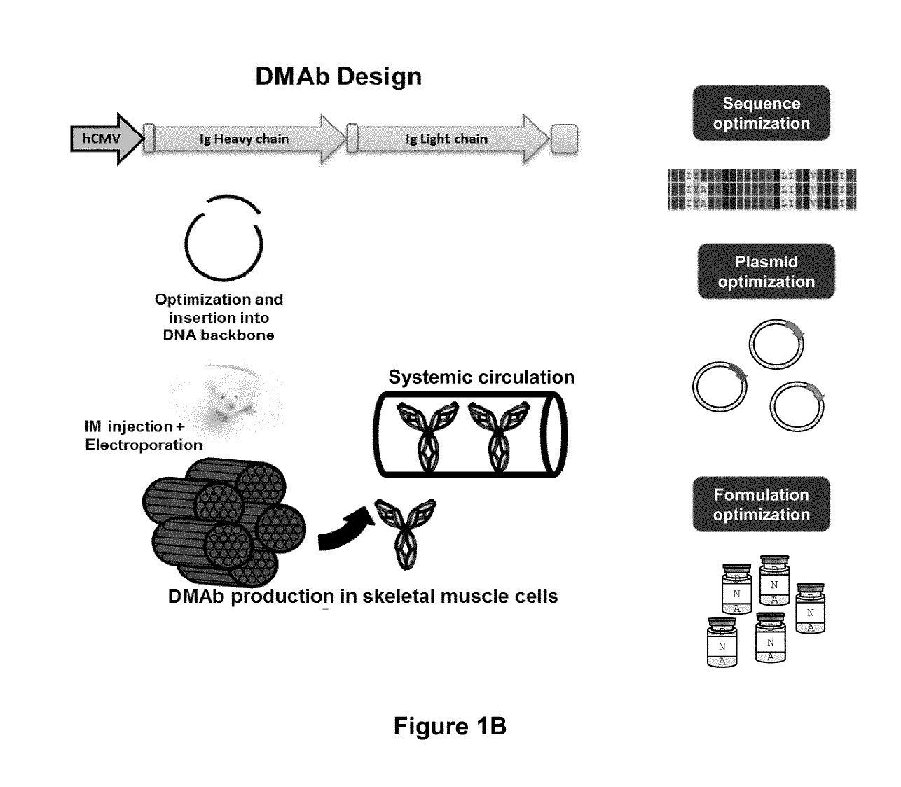

An illustration of DMAb delivery, as compared to traditional protein MAb delivery, is depicted in FIG. 1. For example, FIG. 1A depicts protein MAb production and delivery. HEK293 or CHO cells are transfected with DNA encoding IgG heavy and light chains using optimized methods. Cell supernatants are harvested and purified to obtain purified protein IgG. Protein IgG is delivered to mice by injection. In contrast, FIG. 1B depicts DMAb delivery. DMAb sequence is nucleotide and amino acid optimized before insertion into a highly optimized plasmid backbone. DMAbs are administered directly into mouse muscle by IM injection, followed by electroporation. Mouse muscle expresses, assembles, and secretes functional mAb in vivo at levels detectable in systemic circulation.

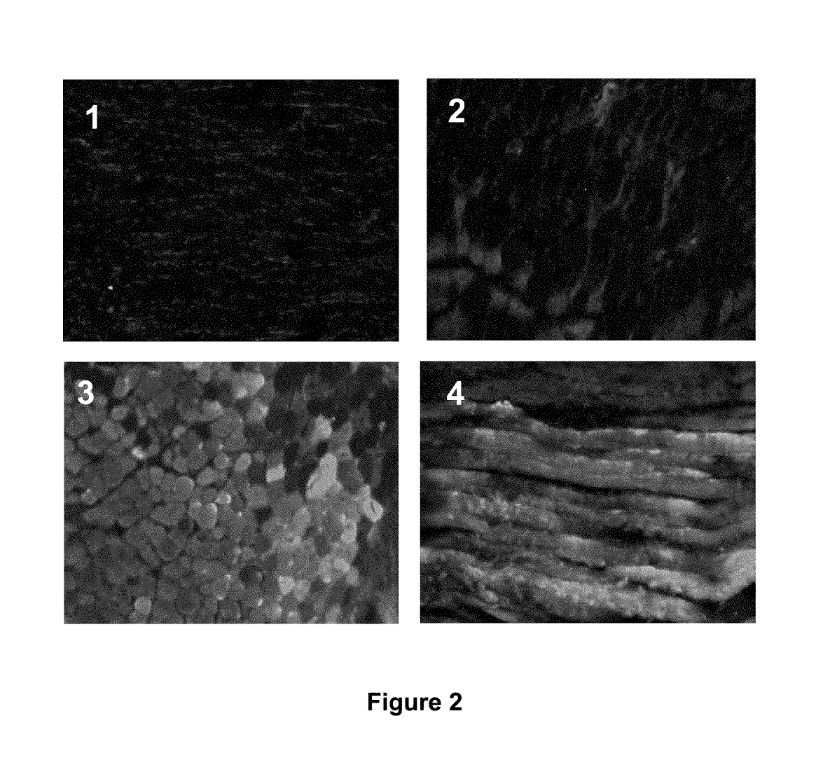

FIG. 2 illustrates the expression of DMAbs in muscle. FIG. 2 depicts immunofluorescence images of sections of the TA muscle treated with DMAb or pGX0001 empty vector backbone delivered with electroporation, and harvested 72 hours later. Human IgG was detected with anti-human IgG followed by a FITC-labelled secondary antibody (green). DAPI stain in blue. Panel 1. No treatment. Panel 2. pVax. Panels 3 & 4. DMAb. Panels 1-3 display a cross-sectional image perpendicular to muscle fibers, and in Panel 4 the image is along the muscle fibers.

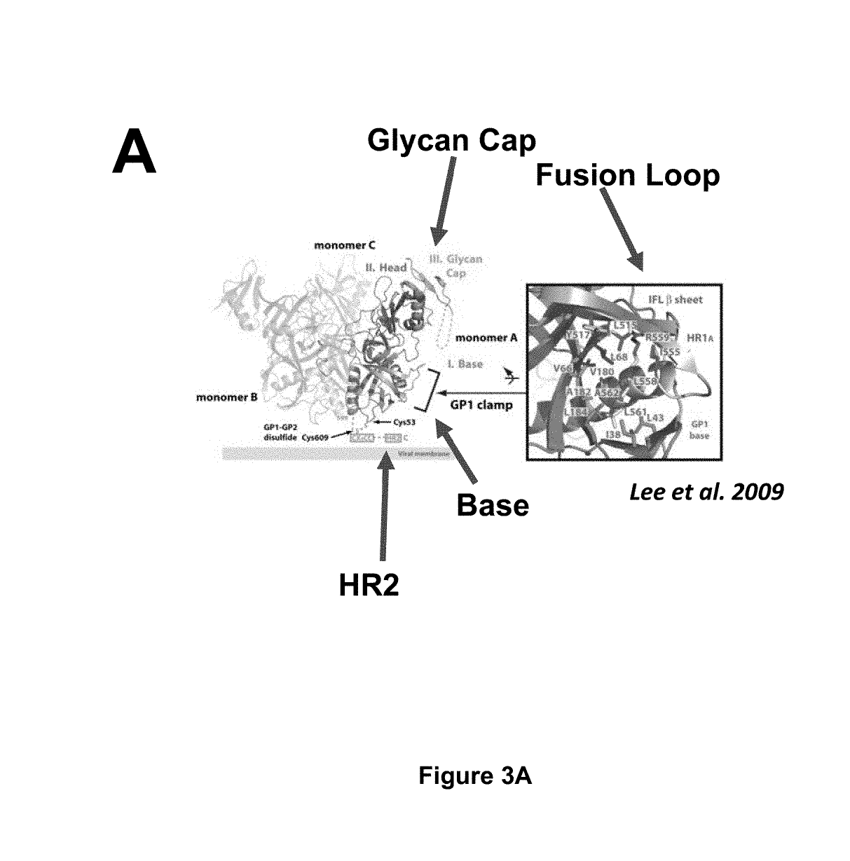

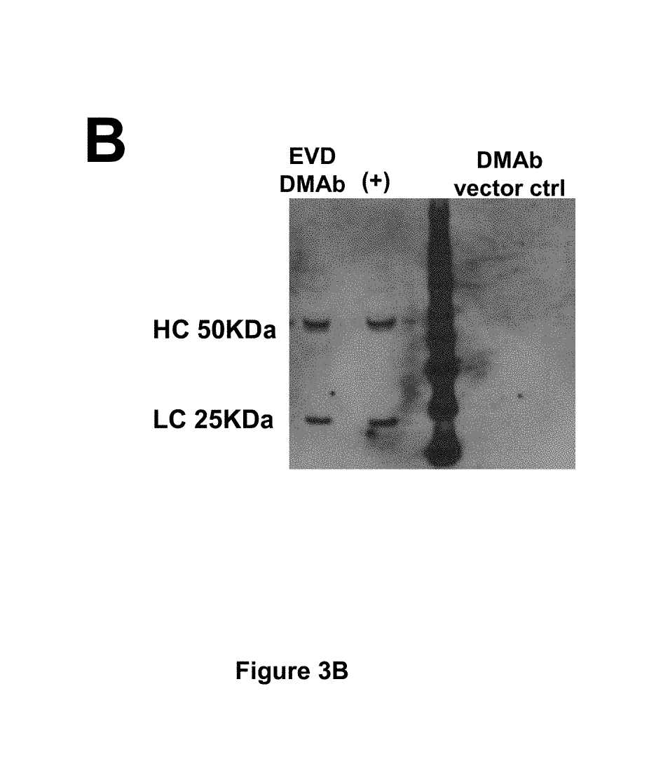

The in vitro expression of Ebola Virus Disease (EVD) DMAbs is shown in FIG. 3. FIG. 3A depicts the structure of the glycan cap (GP) and fusion loop (Lee et al. 2009). HEK293 T cells were transfected with EVD DMAb, and a Western blot was performed to detect the presence of DMAb heavy and light chains. FIG. 3B demonstrates that the heavy and light chains are detected in lysates from EVD DMAb-transfected HEK293T cells.

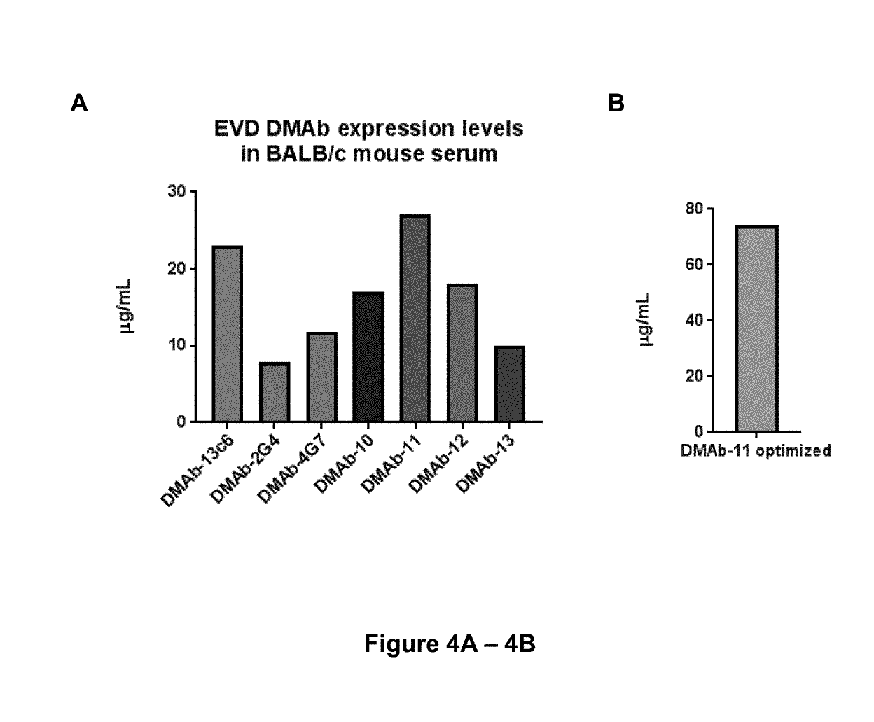

The in vivo expression of EVD DMAbs were investigated in BALB/c mice. BALB/c mice received 400 μg of the various indicated EVD DMAbs by intramuscular injection, followed by electroporation (FIG. 4A). The peak expression level of human IgG1 in the mouse serum was detected, which is depicted in FIG. 4A. As shown in FIG. 4B, optimization of the formulation and delivery of DMAb-11 can enhance DMAb circulating levels.

### Example 2

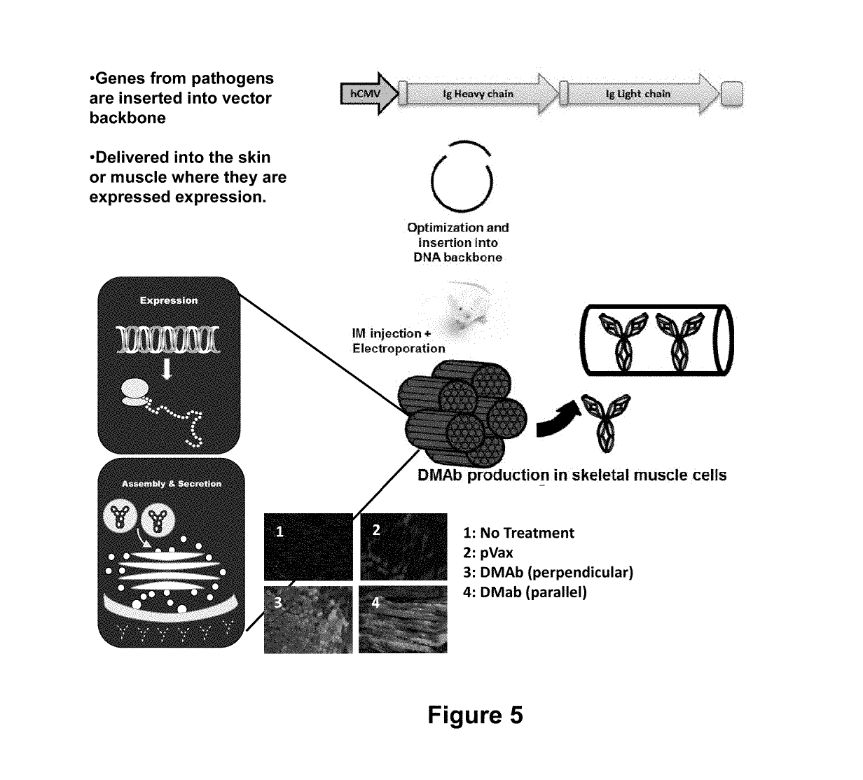

The studies presented herein demonstrate that synthetic DNA monoclonal antibodies (DMAbs) exhibits improved in vivo expression. Genes from Ebola virus were inserted into a vector backbone and delivered into the skin or muscle where they are expressed as synthetic monoclonal antibodies (FIG. 5). As described herein, EVD DMAbs were designed with optimized sequences and have human Fc.

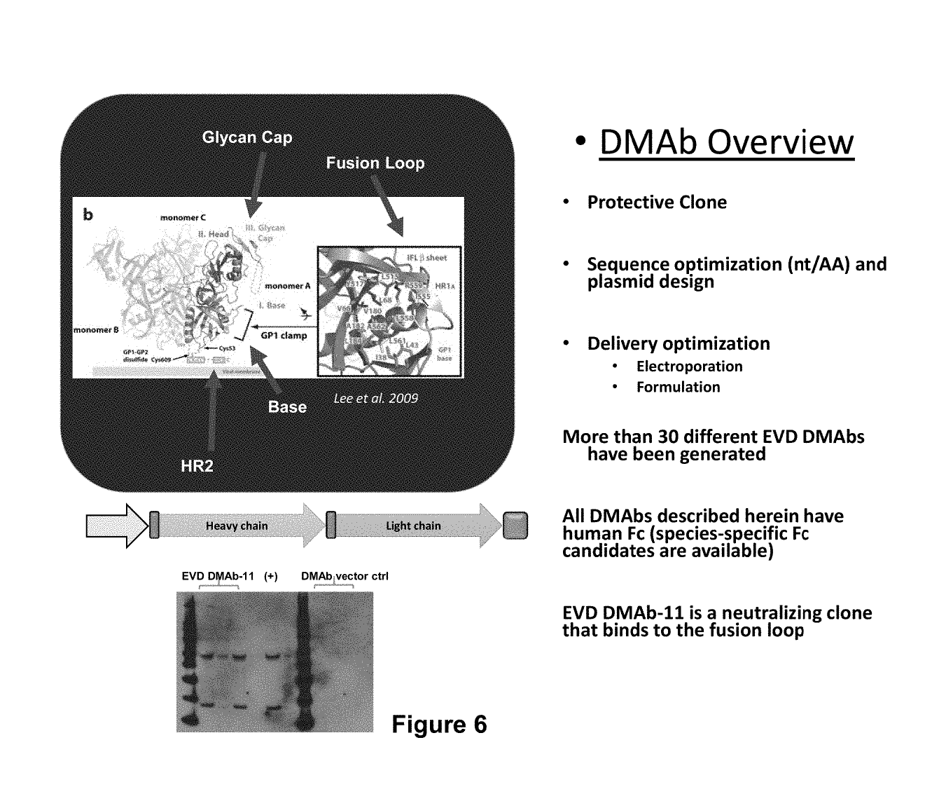

FIG. 6 illustrates the development of EVD DMAbs. While more than 30 EVD DMAbs have been generated, the results presented herein demonstrate that that a representative DMAb, DMAb-11, is expressed in vivo and provides preventative protection against lethal mouse-adapted EBOV challenge.

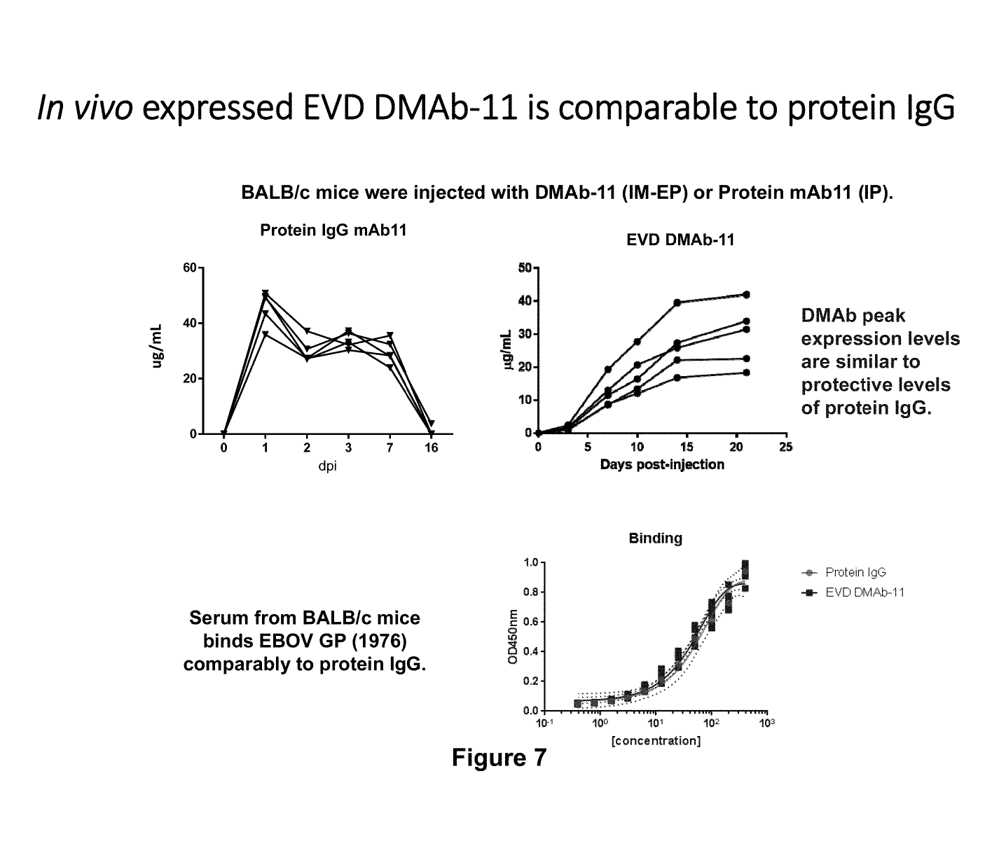

In vivo expressed EVD DMAb-11 has expression levels comparable to protein IgG mAb11. BALB/c mice were injected with DMAb-11 (IM-EP) or Protein mAb11 (IP). DMAb peak expression levels are similar to protective levels of protein IgG (FIG. 7). Further, the serum from BALB/c mice binds EBOV GP (1976) comparably to protein IgG mAb11.

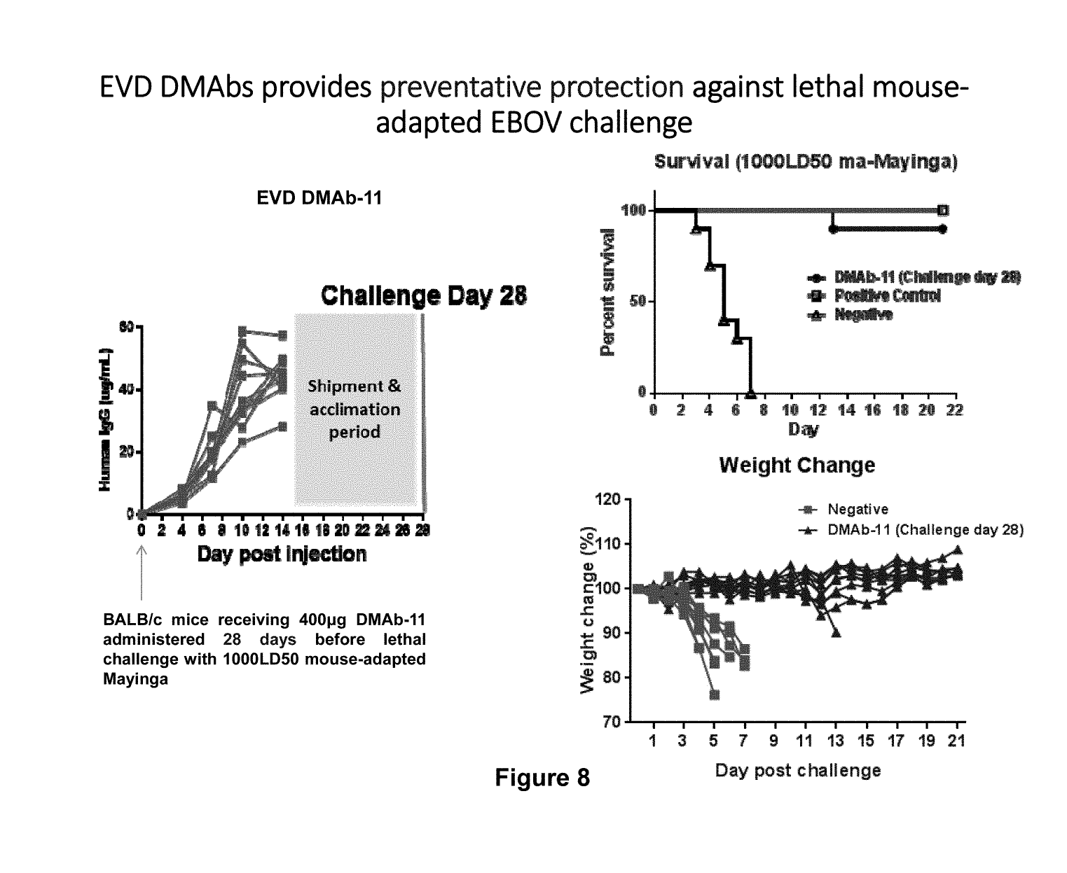

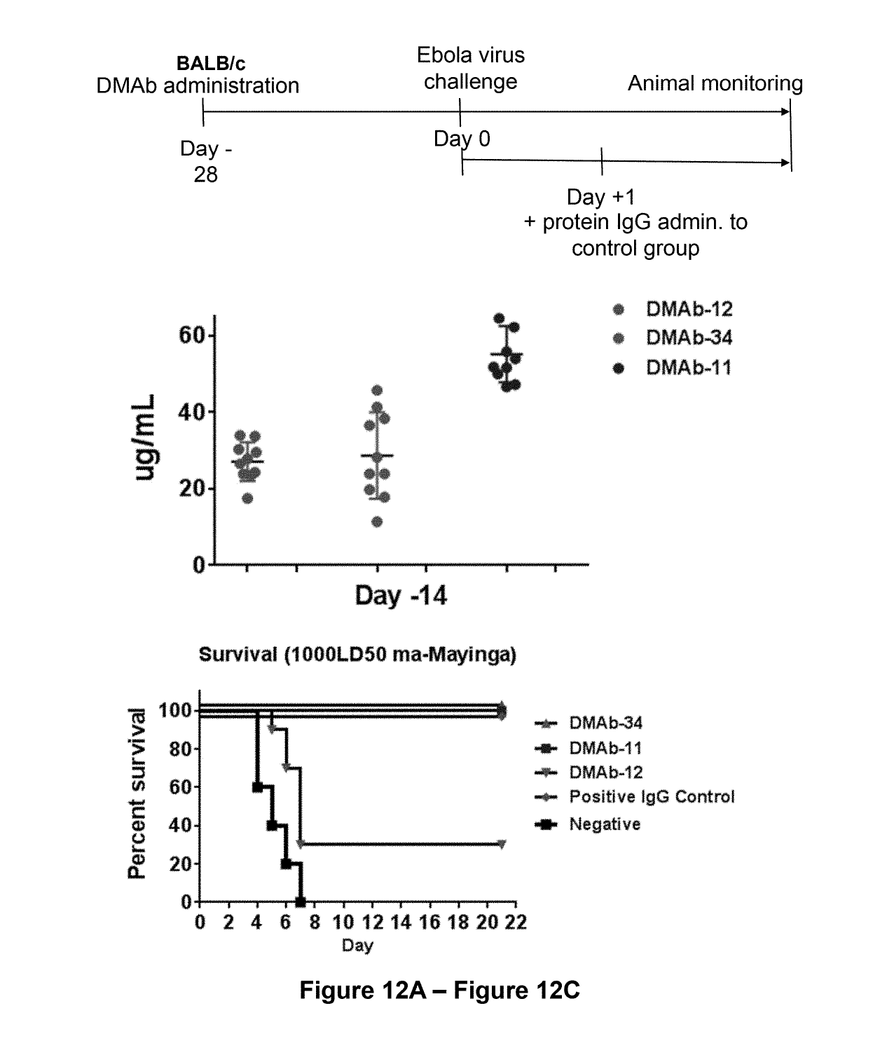

EVD DMAbs provides preventative protection against lethal mouse-adapted EBOV challenge. BALB/c mice receiving 400 μg DMAb-11 administered 28 days before lethal challenge with 1000LD₅₀ mouse-adapted Mayinga. FIG. 8 shows that BALB/c mice which received DMAb-11, survived the lethal challenge, while each of the negative control mice died by day 7. In further illustration of EVD DMAbs preventative protection against mouse-adapted EBOV, the weight change in mice was measured. Negative control mice rapidly lost weight, while the DMAb treated mice maintained their weight (FIG. 8). FIG. 12 shows that DMAb-12, DMAb-13 and DMAb-11 are expressed in vivo 14 days after DMAb injection and improve survival following lethal challenge with 1000LD₅₀ of mouse-adapted Ebola virus compared to negative control mice.

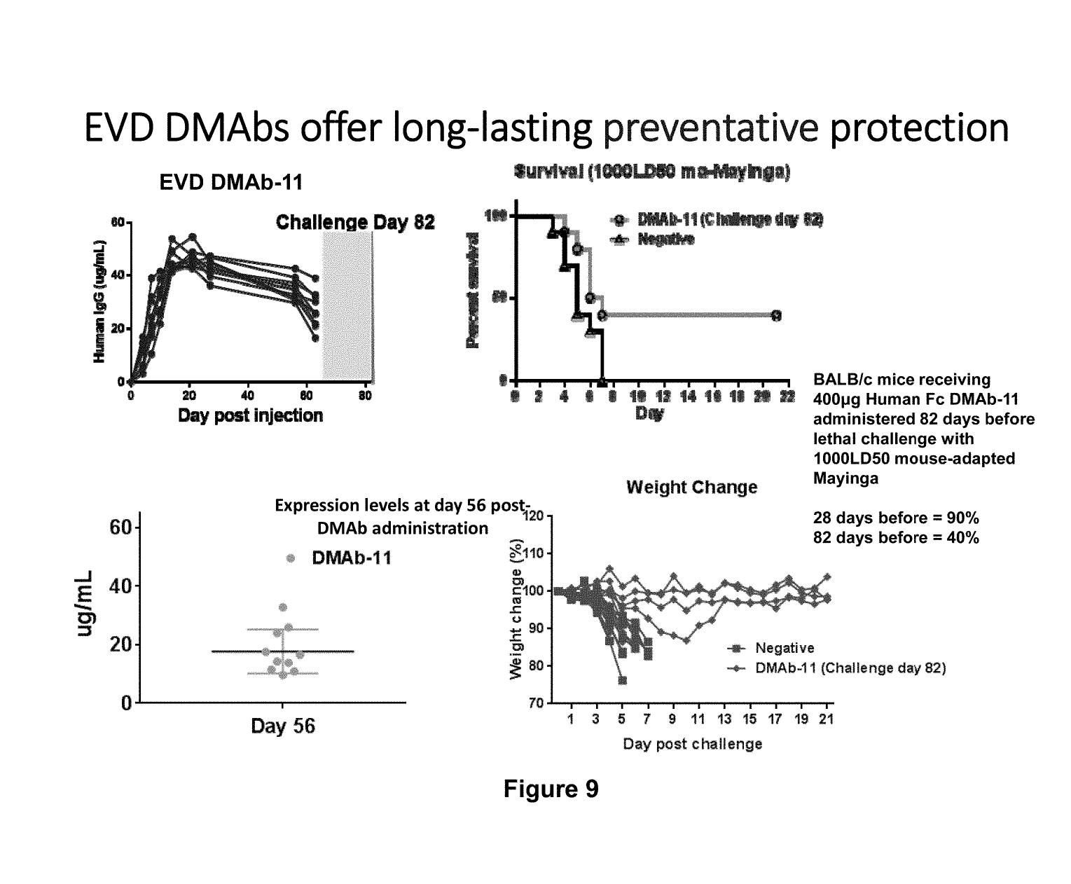

The EVD DMAbs offer long-lasting preventative protection. BALB/c mice receiving 400 μg Human Fc DMAb-11 administered 82 days before lethal challenge with 1000LD₅₀ mouse-adapted Mayinga. FIG. 9 shows that 40% of the BALB/c mice which received DMAb-11, survived the lethal challenge 82 days after DMAb was administered, while each of the negative control mice died by day 7. In further illustration of EVD DMAbs preventative protection against mouse-adapted EBOV, the weight change in mice was measured. Negative control mice rapidly lost weight, while the DMAb treated mice maintained their weight (FIG. 9).

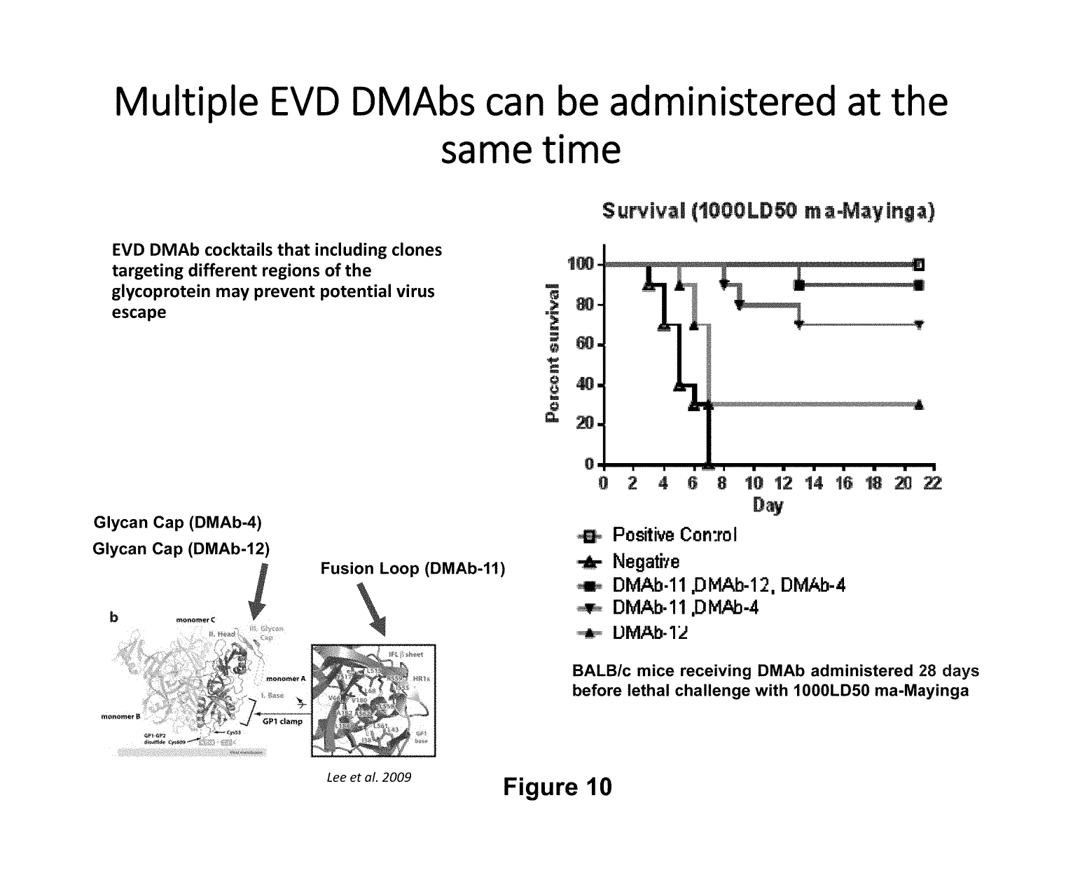

It has been shown that virus escape variants can occur following IgG mAb treatment in non-human primates. Accordingly, EVD DMAb cocktails that including clones targeting different regions of the glycoprotein may prevent potential virus escape. FIG. 10 shows experiments where multiple EVD DMAbs were administered simultaneously. Administering two (DMAb-11 and DMAb-4) or three (DMAb-11, DMAb-12 and DMAb-4) improves survival after lethal challenge with 1000LD₅₀ ma-Mayinga (FIG. 10).

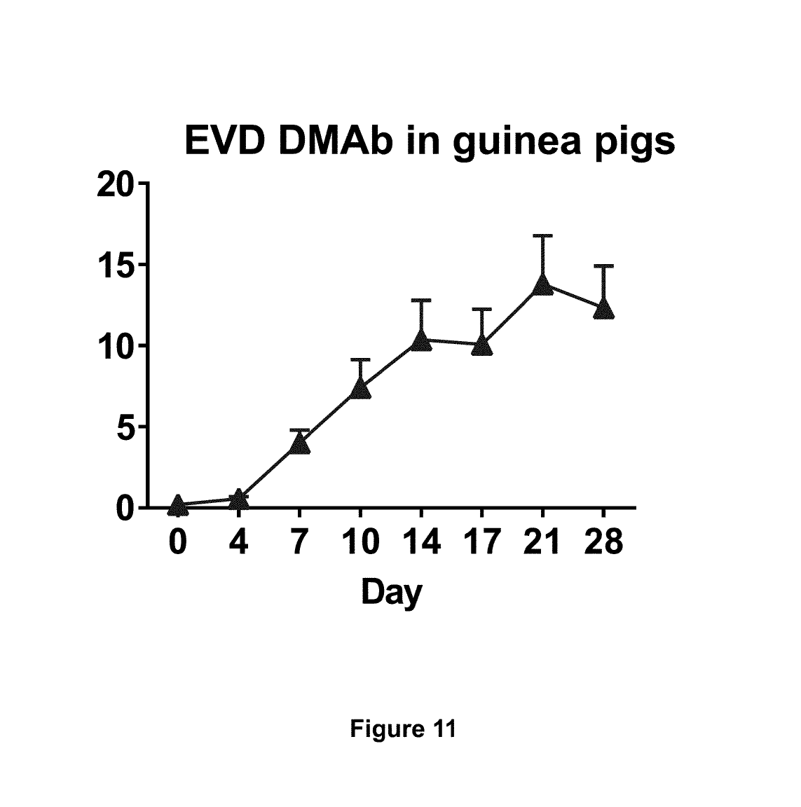

In further demonstrating of the protective effects of EVD DMAbs, expression studies in guinea pigs was carried out. FIG. 11, shows that EVD DMAbs are expressed in guinea pigs. These vaccines rapidly induce a protective state and can shorten window of exposure during vaccination program. This may allow a biologic product bridge for the developing world.

### Example 3

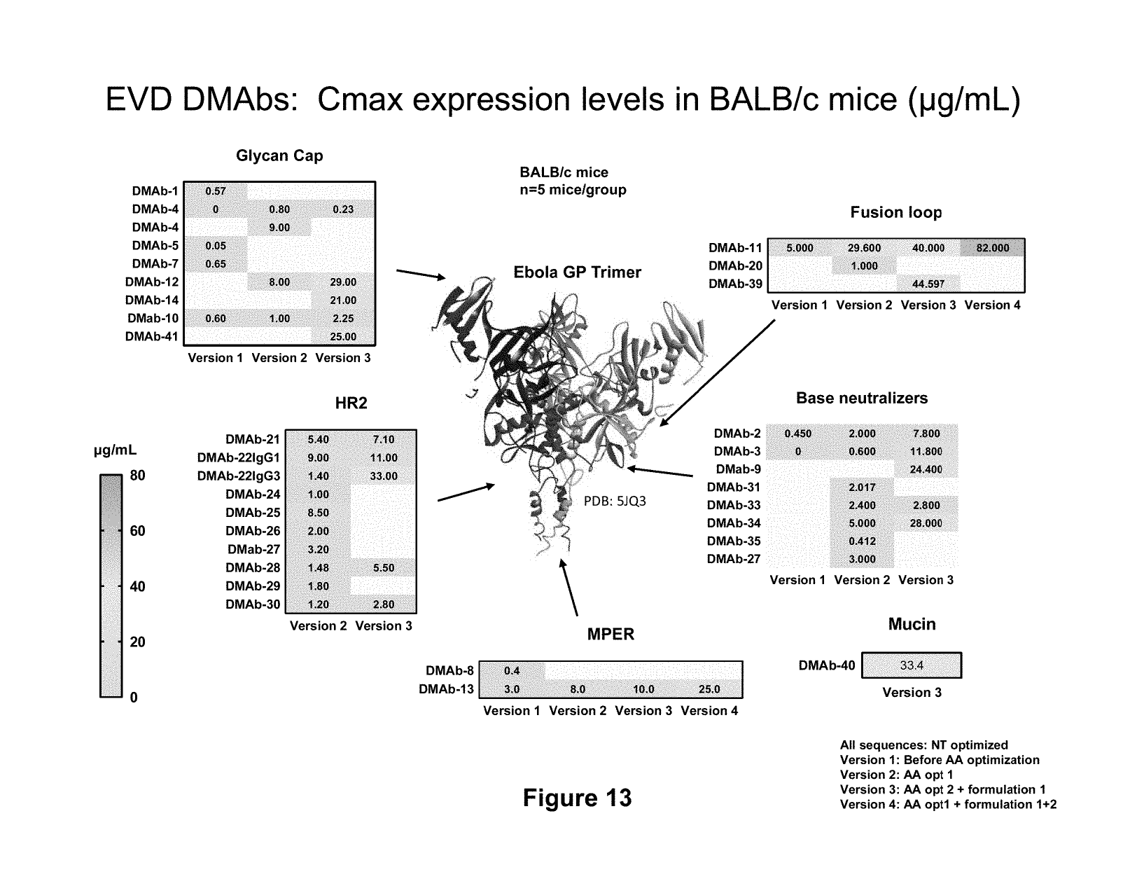

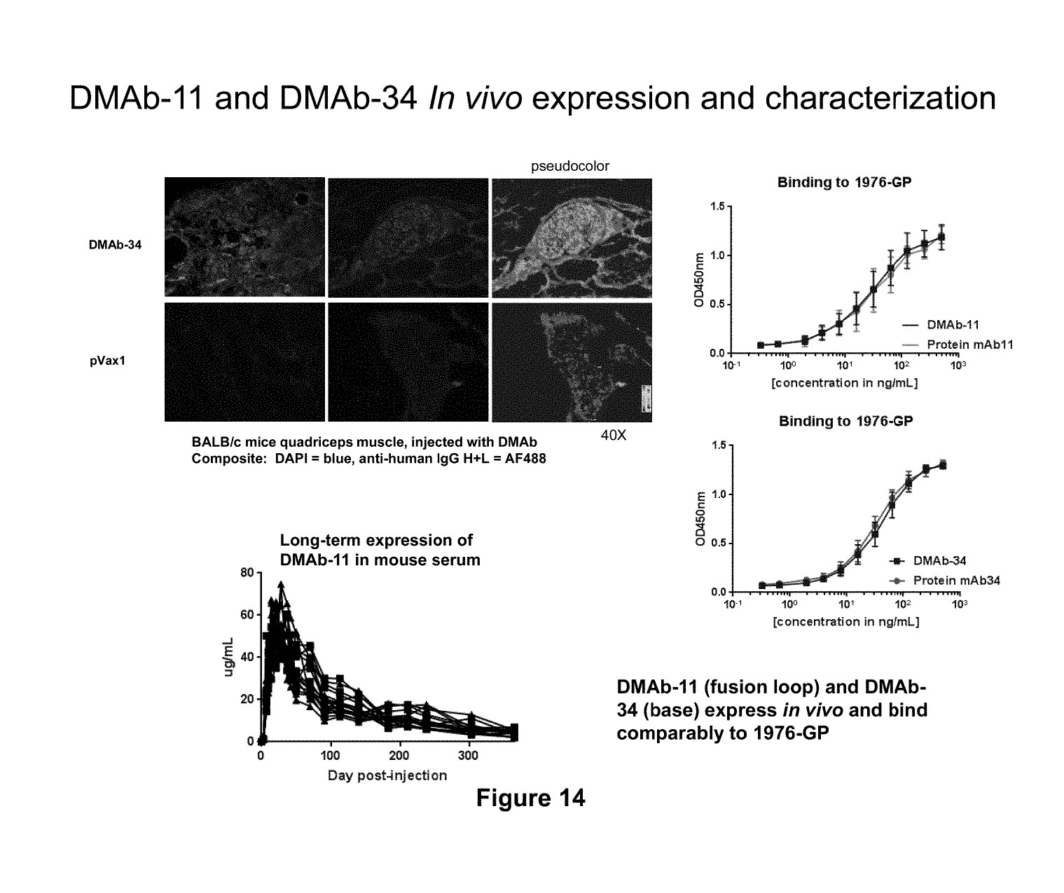

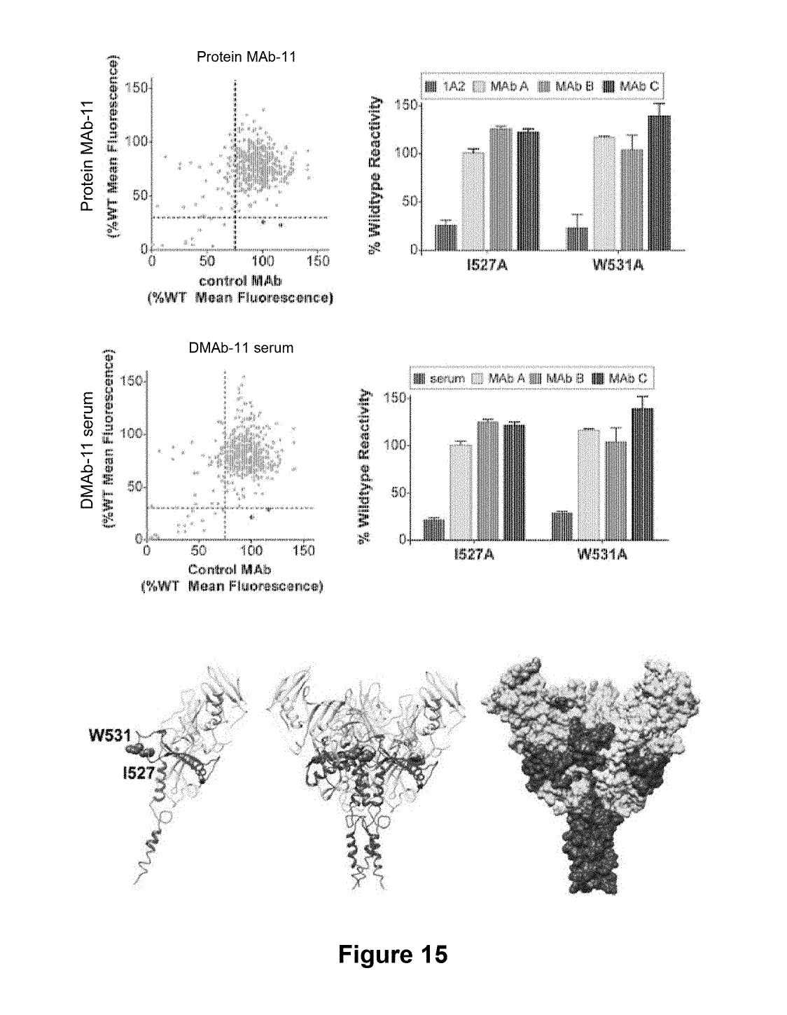

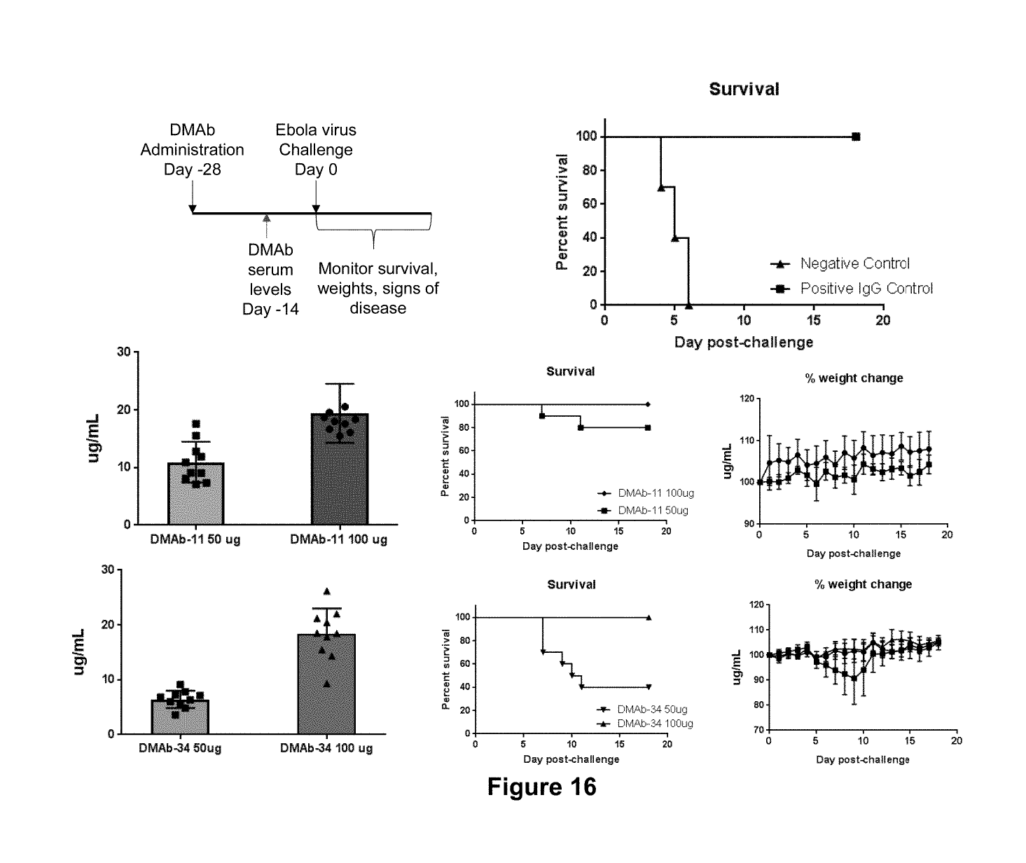

The studies presented herein demonstrate that EVD DMAbs are protective in mice challenged with ma-EBOV. EVD DMAbs are expressed in mice (FIG. 13). DMAb-11 (fusion loop) and DMAb-34 (base) express in vivo and bind comparably to 1976-GP (FIG. 14). Shotgun mutagenesis epitope mapping was performed by alanine scanning of EBOV GP using HEK 293T cells (FIG. 15). Both EVD DMAb-11 and EVD-34 protect against lethal mouse-adapted Mayinga challenge (1000LD₅₀) (FIG. 16).

### Example 4

The data presented herein demonstrates the engineering and development of twenty-seven novel DMAbs delivering potent anti-Zaire Ebolavirus (EBOV) GP clones, twenty-two of which have been isolated from human EVD survivors. Novel sequence modifications using in silico design combined with enhanced DNA formulation development can lead to high levels of anti-GP human IgG DMAb expression in vivo in mouse models. Two anti-GP DMAbs were selected, DMAb-11 (targeting the GP-fusion loop) and DMAb-34 (targeting the GP base), for further demonstration that DMAbs have comparable GP epitope binding to protein Ig. The data demonstrate that in vivo delivery of DMAb-11 and DMAb-34 GP-DMAbs results in consistent human Ig mAb production and are protective individually as well as in combination against lethal mouse-adapted EBOV challenge in mice. Taken together, the results presented herein demonstrate that facilitated DMAb delivery is a promising approach for in vivo production of fully human anti-EBOV mAb clones the data support translation into larger models.

The current study was designed to evaluate the potential for novel synthetic DNA-encoded monoclonal antibodies, DMAbs, to deliver potent mAbs isolated from EVD survivors targeting the Zaire ebolavirus glycoprotein. It was first hypothesized that the DMAb sequence has significant impact on in vivo expression levels and that these levels are independent of in silico prediction algorithms typically used for bioprocessed mAb manufacturing. To accomplish this, the described experiments assessed the impact of in silico design on in vivo human DMAb expression pharmacokinetics and characterized DMAbs in comparison with bioprocessed mAb. Based on this data, it was hypothesized that optimized anti-GP DMAbs encoding mAbs from EVD survivors can provide pre-exposure protection against lethal mouse-adapted EBOV challenge.

Samples Size and Power Calculations

Sample size and power analysis calculations were performed in SPSS Statistics software (IBM). For in vivo DMAb expression experiments in mice, sample sizes were calculated for two independent proportions, α=0.05 and minimum power of 0.90. Based on these parameters, a minimum of five mice per group was calculated to be necessary in order to achieve adequate power in the experiments. For survival studies, a minimum sample size of n=10 was necessary

Endpoint Determination

Anti-GP DMAb expression levels in mice were followed from day 0 until no more human IgG1 was detected in mouse serum. For survival experiments, endpoints were determined as a terminal point due to course of disease or at day 21 post-challenge, once all surviving animals had recovered. Animals were euthanized if they lost more than twenty percent starting body weight or reached a pre-defined clinical score.

Replicates

All in vitro transfections were performed with three technical replicates. One independent repeat was performed for all transfections. In vivo experiments for DMAb expression pharmacokinetics in mice were performed once in order to minimize the use of animals. Following analysis of the data, the top expressing DMAbs were selected for repeats to increase statistical power and demonstrate the consistency of the in vivo DMAb approach. DMAb-11 expression was repeated in at least six independent studies and three independent protection studies. DMAb-34 expression was repeated in at least four independent studies and two independent protection studies.

Experimental Design

Controlled laboratory experiments were used to evaluate all the anti-GP DMAbs described in this manuscript. These included studies evaluating pharmacokinetic levels of human IgG circulation in mouse serum following human DMAb administration, comparison studies with control protein MAb (binding, epitope mapping, neutralization), and protection studies evaluating different anti-GP DMAb doses in a mouse lethal challenge model.

Human IgG Quantification by ELISA

Ninety-six well, high-binding immunosorbent plates were coated with 1 μg mL⁻¹ purified anti-Human IgG-Fc and incubated overnight at 4° C. On the next day, plates were blocked with PBS containing 10% FBS for 1 hour at room temperature. Plates were washed with PBS containing 0.05% Tween-20 in between each incubation steps. Plates were incubated with a standard and samples for 1 hour at room temperature. Purified Human IgG/Kappa was used as a standard. Samples were diluted in PBS containing 1% FBS and 0.02% Tween-20. Following the incubation, samples were probed with anti-human Kappa light chain antibody conjugated to horseradish peroxidase in 1:20,000 dilution and incubated for 1 hour at room temperature. After incubation, plates were developed with o-Phenylenediamine dihydrochloride (OPD) substrate for 25 minutes in the dark and stopped with 2N H₂SO₄. A BioTek Synergy2 plate reader was used to read plates at OD 450 nm.

Binding ELISA

Ninety-six well, high-binding immunosorbent plates were coated with 1 μg mL⁻¹ Ebola virus Glycoprotein (strain Mayinga 1976) and incubated overnight at 4° C. Alternatively, Ebola virus Glycoprotein (strain H. sapiens-wt/GIN/2014/Kissidougou-C15) was used. On the next day, plates were blocked using PBS containing 5% non-fat milk and 0.02% Tween-20 for 90 minutes at 37° C. Plates were washed with PBS containing 0.05% Tween-20 in between each incubation steps. After being blocked, plates were incubated with samples in series of dilution for 1 hour at 37° C. Following incubation, samples were probed with anti-human IgG (H+L) conjugated to horseradish peroxidase for 1 hour at 37° C. Plates were developed using OPD substrate for 25 minutes in the dark and stopped using 2N H₂SO₄. A BioTek Synergy2 plate reader was used to read plates at OD 450 nm.

Western Blot

Cell lysate was collected in transfected cells in cell lysis buffer. Samples were centrifuged at 16,000×g and the supernatant containing the protein fraction was collected. Total protein concentration of each sample was measured using Bicinchoninic acid (BCA) assay. Samples were reduced for 10 minutes at 70° C. using NuPAGE™ Sample Reducing Agent (10×). 10 μg of samples were loaded on a NuPAGE™ 4-12% Bris-Tris gel SeeBlue™ Pre-stained Protein Standard was used as standard markers. The gel was transferred to a PVDF membrane Immobilon-FL using iBlot™ 2 system. The membrane was blocked with Odyssey® Blocking Buffer in PBS for 1 hour. Beta Actin Monoclonal Antibody in 1:5,000 dilution was added as a positive control. Following incubation, the membrane was incubated with Anti-Human IRDye 800CW in OBB containing 0.1% Tween-20 and 0.01% SDS for 1 hour in the dark. Alternatively, anti-Mouse IRDye 680RD was used as a secondary antibody. After being washed with PBS, the membrane was scanned using Odyssey® CLx Imager.

Neutralization Assay

Neutralization assays were performed using live Zaire Ebola virus expressing enhanced green fluorescent protein (eGFP). The day before the assay, Vero E6 cells were plated in ninety-six well black plates with a transparent bottom. Serum from DMAb-administered mice was serially diluted two-fold in DMEM down a ninety-six well plate and incubated with 0.1 tissue culture infectious dose fifty (TCID50) virus/cell for one hour at 37° C., 5% CO₂. The serum:virus mixture was then added to Vero E6 cells (85-90% confluent) and incubated for one hour at 37° C., 5% CO₂. After incubation, the mixture was removed and 100 μl of DMEM plus 2% Bovine Growth Serum. Cells were then incubated for seventy-two hours at 37° C., 5% CO₂. Plates were read for GFP fluorescence from the bottom using a BioTek Synergy HT plate reader.

Shotgun Mutagenesis Epitope Mapping

Shotgun Mutagenesis epitope mapping (Davidson et al., 2014, Immunology 143:13-20) on EBOV-GP. Briefly, alanine scanning mutagenesis was carried out on an expression construct for EBOV-GP (strain Mayinga-76; UniProt accession # Q05320) lacking the mucin-like domain (residues 311-461). Residues 33-310 and 462-676 of the EBOV delta (A) mucin GP were mutagenized to create a library of clones, each with an individual point mutant. Residues were changed to alanine, with alanine residues changed to serine. GP residues 1-32, which constitute the GP signal peptide, were not mutagenized. The resulting EBOV GP alanine-scan library covered 492 of 493 of target residues (99.9%). Each mutation was confirmed by DNA sequencing, and clones were arrayed into 384-well plates, one mutant per well. Each library plate also contained negative control wells with vector alone, and positive control wells containing wild-type EBOV Amucin GP.

Before epitope mapping on the mutation library, it was confirmed that MAb 5.6.1A2 and 15784 and mouse DMAb-11 and DMAb-34 serum showed reactivity with EBOV-GP, and identified an appropriate MAb concentration and serum dilution for screening the mutation library. MAb 5.6.1A2 and 15784 and pooled DMAb-11 and DMAb-34 mouse serum were tested for binding to wild-type EBOV Amucin GP expressed in HEK-293T cells. After addition of a fluorescent secondary antibody, the mean cellular fluorescence was detected using an Intellicyt flow cytometer. The entire EBOV Amucin GP library expressed in HEK-293T cells was screened for binding of mutant clones to MAb 5.6.1A2 and 15784, or to DMAb-11 and DMAb-34 mouse serum, by detecting mean cellular fluorescence. Mutations within clones were identified as critical to the MAb epitope if they did not support reactivity of the MAb, but did support reactivity of other conformation-dependent MAbs. This counter-screen strategy facilitates the exclusion of GP mutants that are globally or locally misfolded or that have an expression defect. Validated critical residues represent amino acids whose side chains make the highest energetic contributions to the MAb-epitope interaction.

GP-DMAb Engineering and Delivery

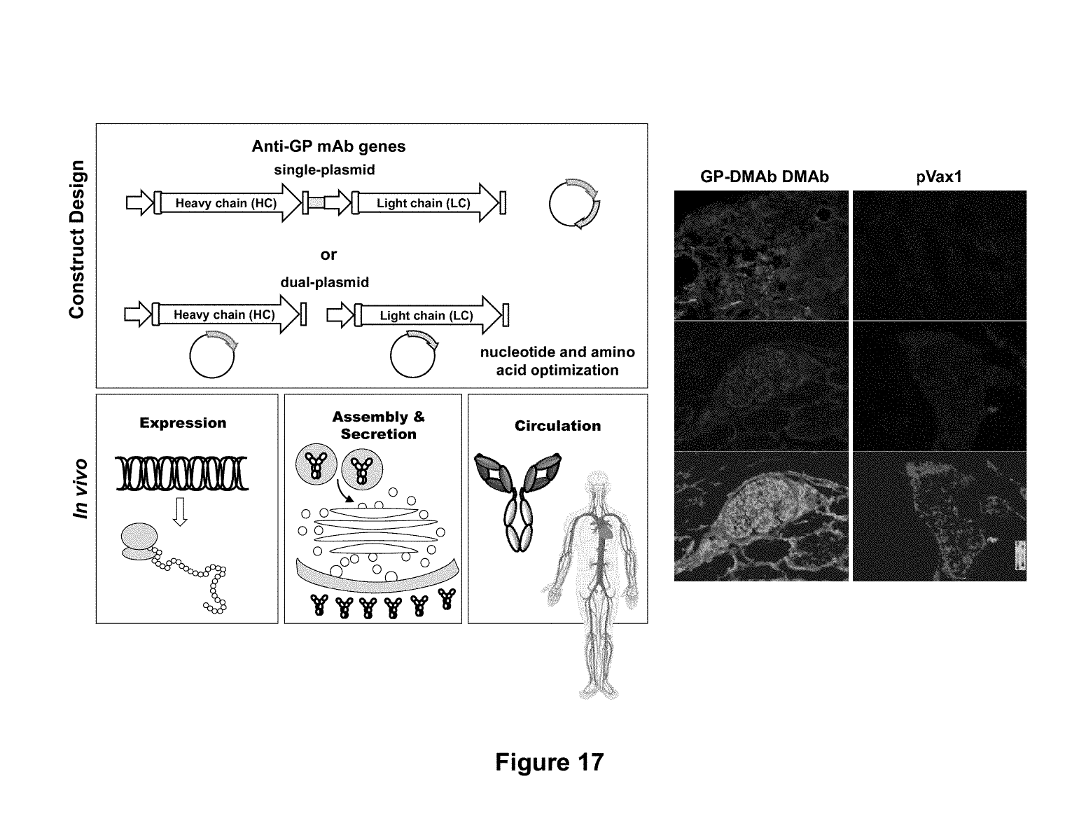

Twenty-seven different anti-EVD mAb clones that target the Ebola virus GP glycan cap, fusion loop, chalice base, HR2 region, and mucin-like domain were selected for development into DMAbs. The sequences of the immunoglobulin (Ig) heavy and light chains were analyzed in silico and optimized to reduce potential RNA secondary structure and for mouse and human codon bias to increase translation efficiency. The full length heavy and light chains were each encoded into a single modified-pVax1 DNA expression vector plasmid, separated by furin and T2A peptide cleavage sites (single-plasmid), or encoded as two separate plasmid constructs dual-plasmid) (FIG. 17A). The DMAb single construct or equal ratio (μg) of heavy and light chain (HC/LC) plasmids were administered in vivo intramuscular injection followed by facilitated electroporation (IM-EP). This resulted in DMAb expression and secretion directly into systemic circulation. Quadriceps muscle slices from mice injected with a GP-DMAb were harvested and stained for human IgG (FIG. 17B) indicating expression of DMAb in muscle cells and fibers (AF488=human IgG, Blue=DAPI).

GP-DMAb Optimization

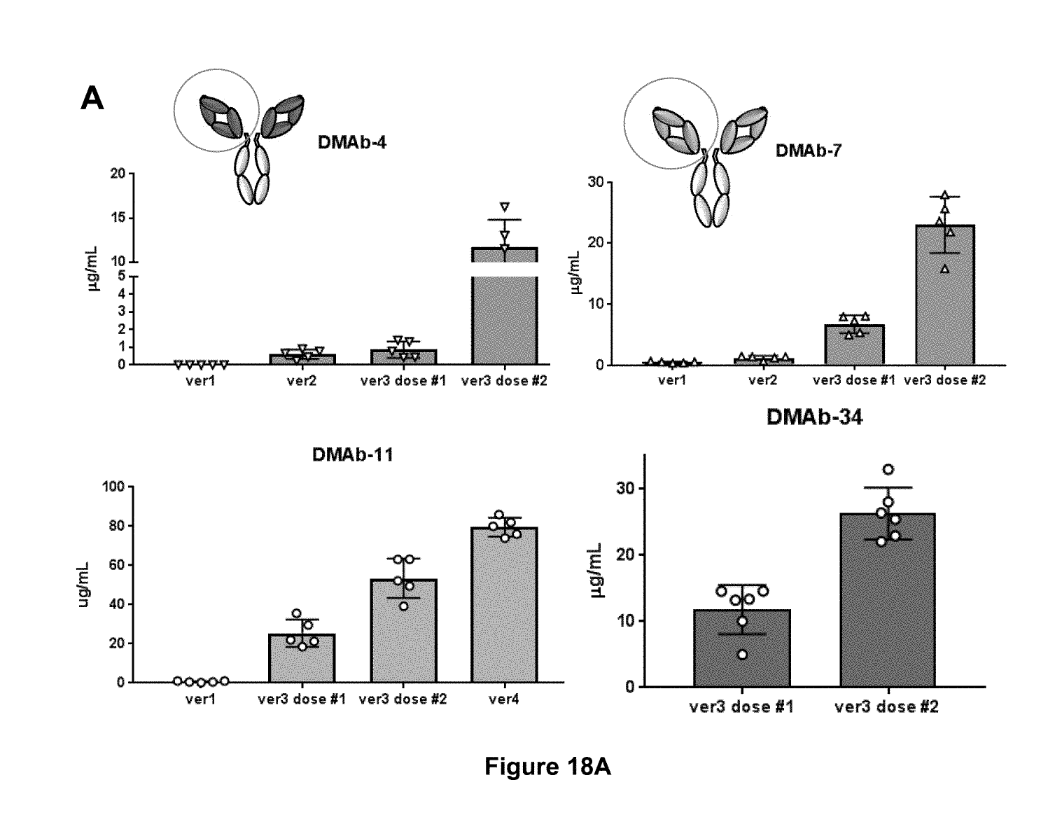

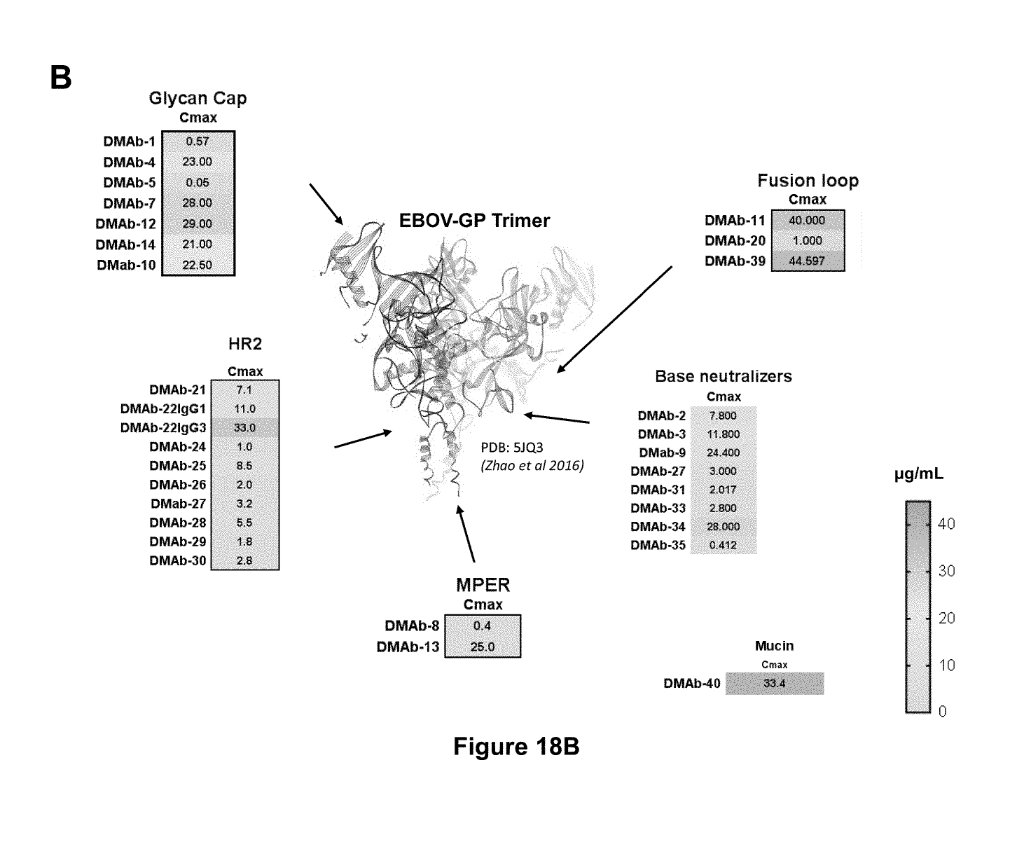

It is well-known that sequence liabilities of IgG can limit bioprocessed mAb production, frequently leading to discarding of an otherwise highly potent mAb clone. In certain cases, it was necessary to further engineer DMAbs through modification of framework amino acids in order to stabilize the immunoglobulin molecule. The mAb genes for clones 4G7 (DMAb-4), 13c6 (DMAb-7), 5.6.1A2 (DMab-11), and 15784 (DMAb-34) were optimized in several ways (FIG. 18). Clones 4G7 and 13c6 are two clones found in the ZMapp cocktail and were isolated from vaccinated mice. Clone 5.6.1A2 was isolated from a 2014-EVD survivor that was treated at Emory University. This clone was isolated from an EVD survivor at the 6-month time point post-treatment. Clone 15784 was isolated among hundreds of survivor-derived mAb clones from a different 2014-EVD patient (Bornholdt et al., 2016, Science 351:1078-83).

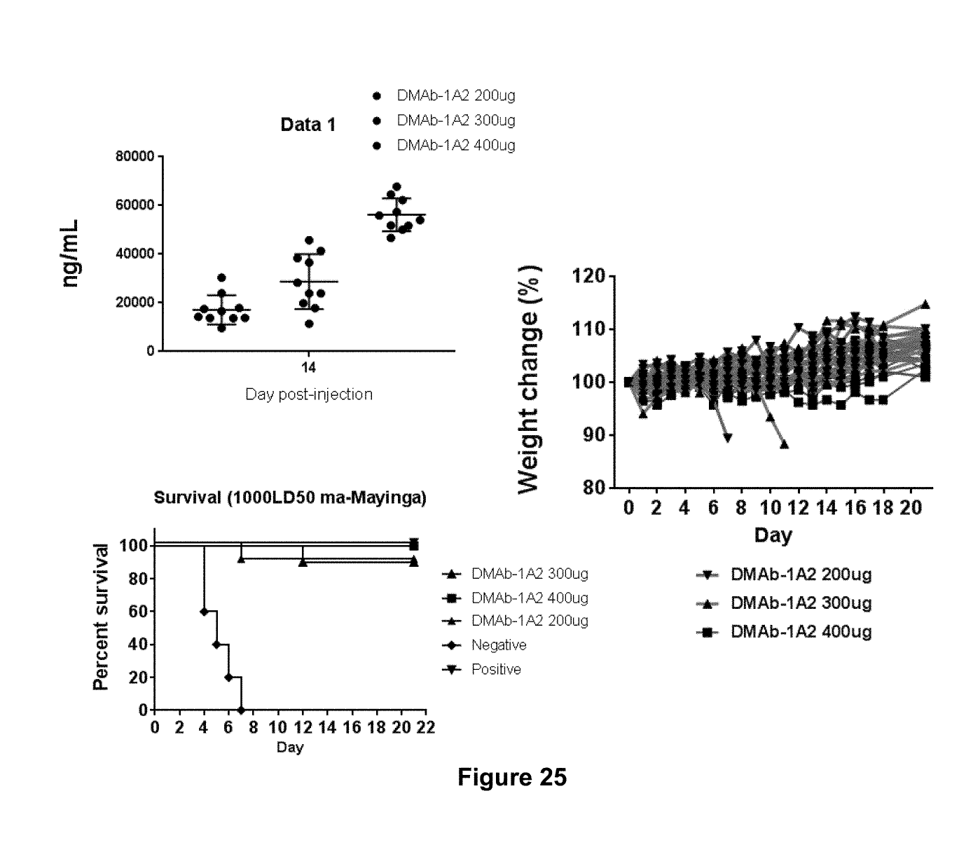

GP-DMAbs were evaluated with only nucleotide optimizations (FIG. 18A, version 1) and amino acid optimizations (FIG. 18A, version 2). For initial experiments, DMAb injection site was pre-treated with hyaluronidase (HYA), version 3 dose #1 (50 μg) and dose #2 (200 μg). HYA digests hyaluronan in the extracellular matrix (ECM), enabling better DNA entry into cells (McMahon et al., 2001, Gene Ther 8:1264-70; Chiarella et al., 2014, Methods Mol Biol. 1121:315-24). However, pre-treatment is not practical for supporting downstream translation to larger models or humans, therefore studies were performed evaluating approaches to co-formulate DNA with HYA for a single injection delivery. Using a previously described anti-Pseudomonas DMAb, DMAb-V2L2 (Patel et al., Nat. Commun., 2017, 8(1):637), it is demonstrated that DMAb co-formulation expresses at comparable in vivo levels to pre-treatment (FIG. 25). Finally, DMAb version 4 (FIG. 18A, Version 4) was designed based on co-formulation of DMAb-11 with hyaluronidase and the anti-inflammatory glucocorticoid methylprednisolone acetate to reduce injection site inflammation. Different variations of these optimizations were performed for 27 different anti-EVD DMAbs (FIG. 18B) and new conditions were applied as later GP-DMAb candidates were being developed.

Expression of DMAb-11 and DMAb-34

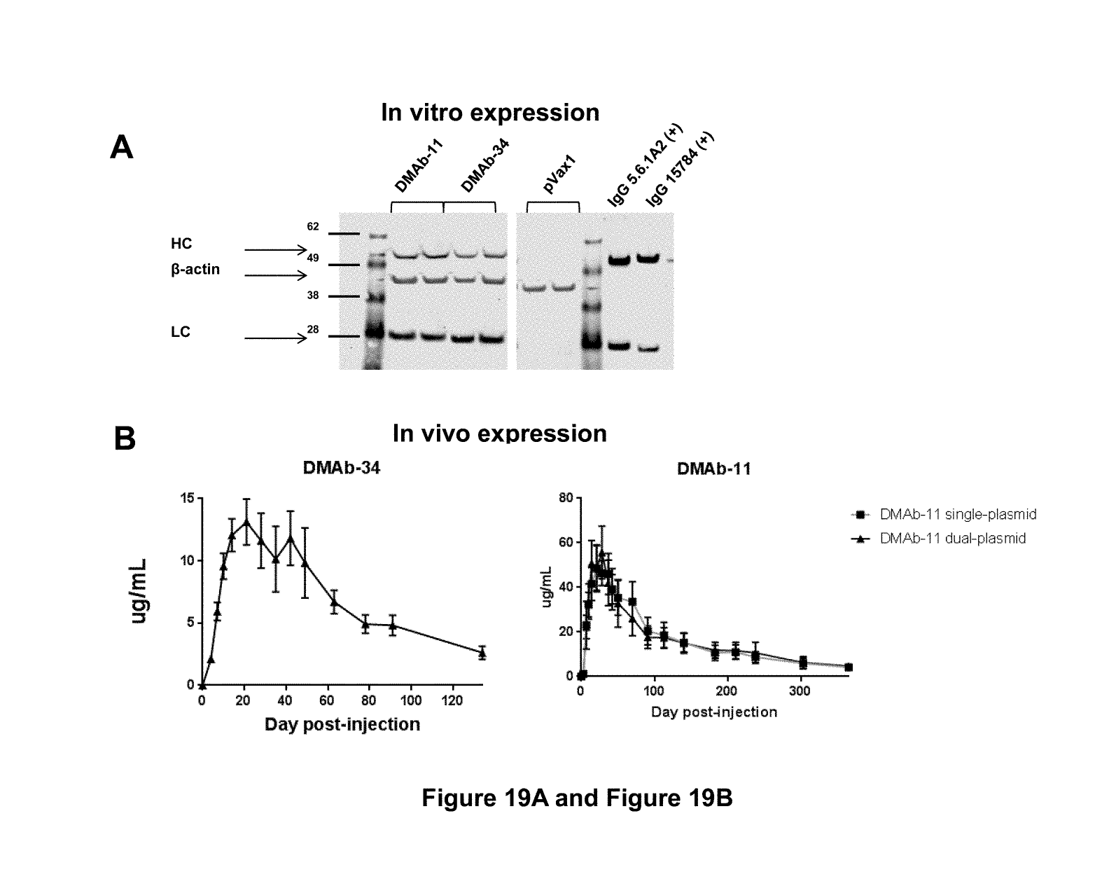

In vitro expression of DMAb-11 and DMAb-34 was quantified in cell lysates of human embryonic kidney (HEK) 293T cells (FIG. 19), harvested 40 hours after transfection. The expected band sizes for the heavy and light chains of DMAb-11 and DMAb-34 observed at approximately 50 kDa and 25 kDa, respectively. The banding pattern for each antibody was comparable to those observed for protein counterpart mAbs 5.6.1A2 and 15784. DMAb-11 encoded as a single-plasmid or dual-plasmid constructs (FIG. 19) was administered to BALB/c mice (n=8-9 mice/group) and followed for 365 days following administration. Long-term expression at high levels was observed and administration of a single-plasmid or dual-plasmids did not impact overall expression kinetics. Dual-plasmid DMab-34 was expressing for at least 168 days.

Comparison of DMAb-11 and DMAb-34 with Protein IgG 5.6.1A2 and 15784

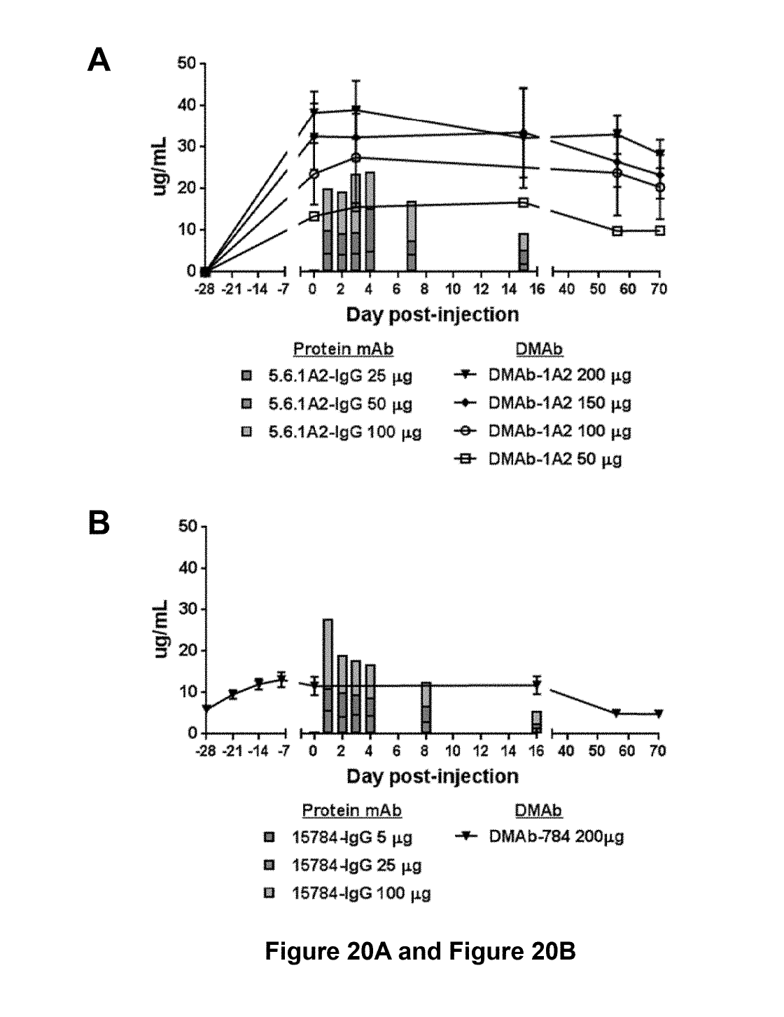

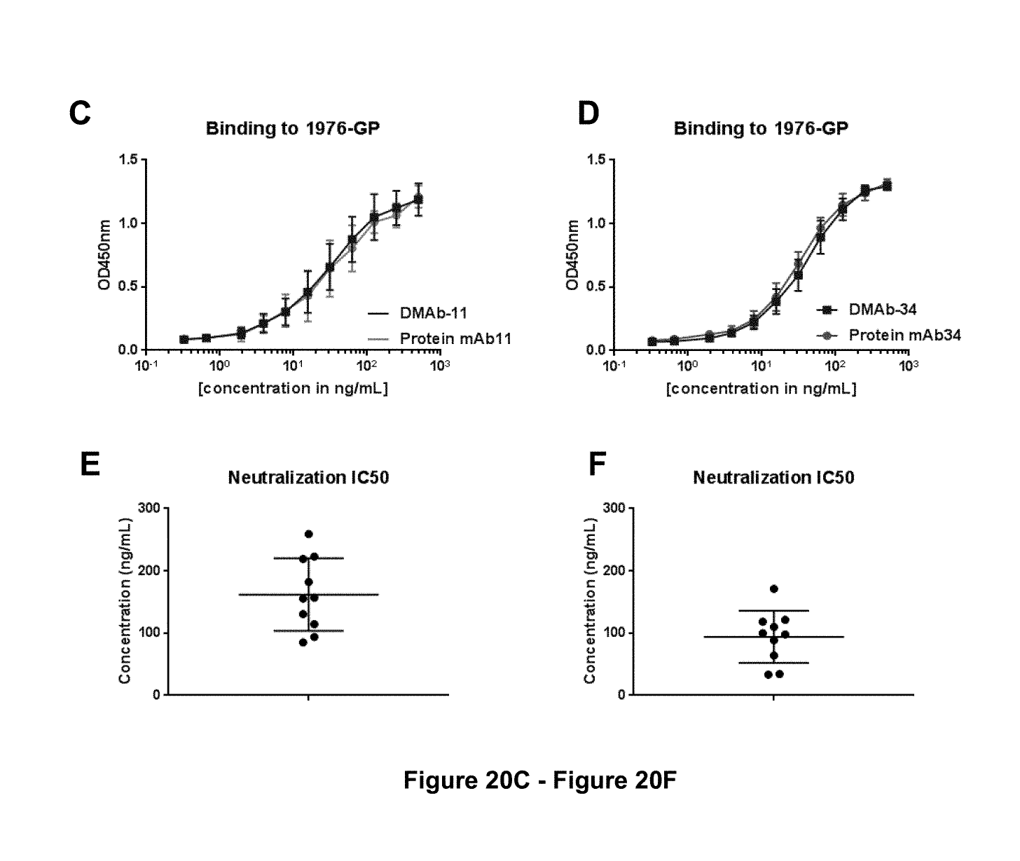

Long-term expression of different doses of DMAb-11 (dual-plasmid, 25-100 μg total DNA) or DMAb-34 (dual-plasmid, 200 μg total DNA) were monitored in parallel with single injection of different doses of protein 5.6.1A2 or 15784 (25 μg-100 μg) (n=5 mice/group) (FIGS. 20A and 20B). Both DMAb-11 and DMAb-34 bind to 1976 EBOV-GP (strain Mayinga) comparably to protein mAb (FIGS. 20C and 20D). Both DMAbs also neutralized live EBOV-GFP (strain Mayinga) virus in a neutralization assay (FIGS. 20E and 20F).

GP-DMAb Epitope Mapping

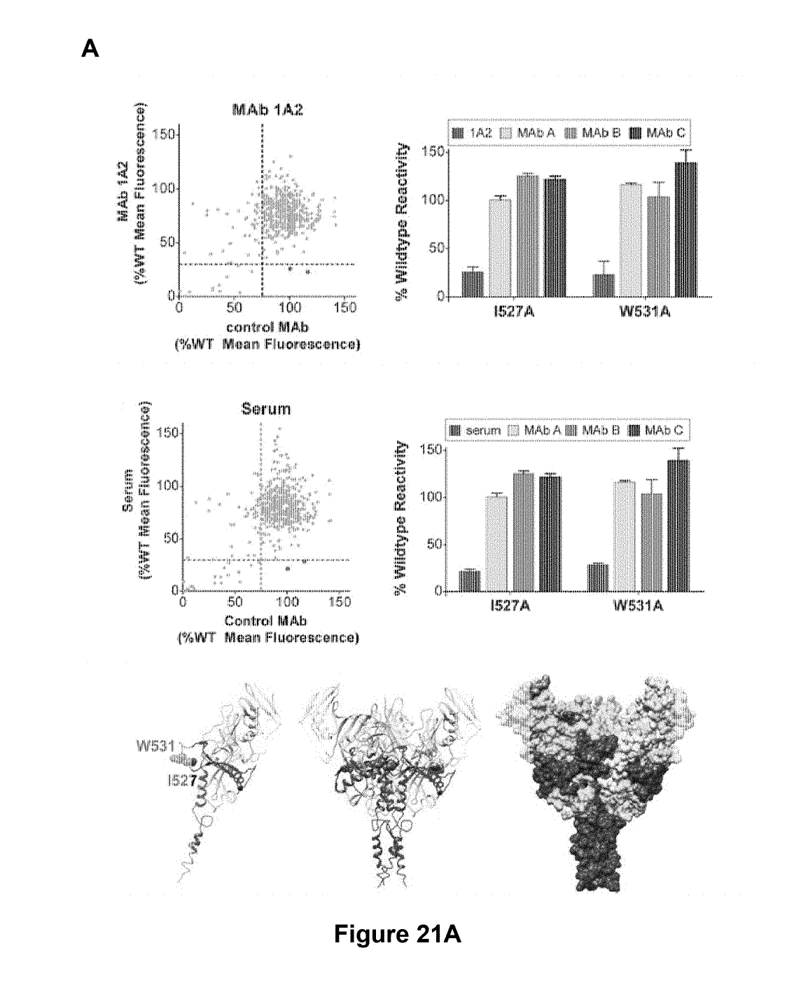

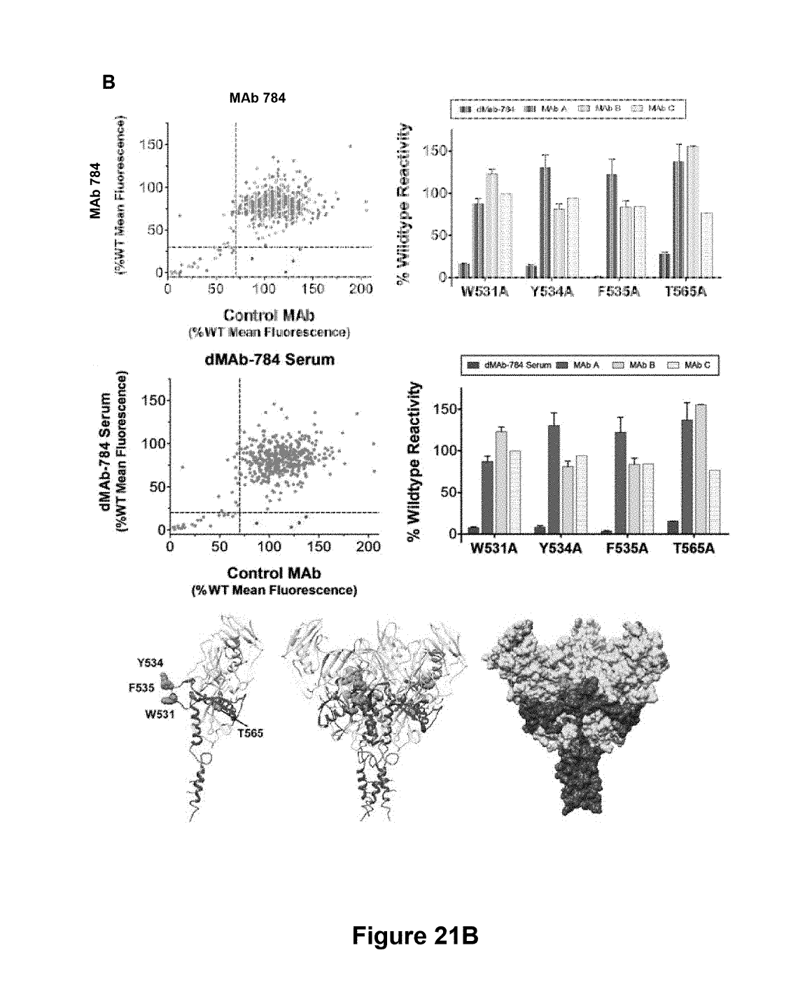

To further address the question of GP-DMAb equivalency to protein IgG, shotgun mutagenesis epitope mapping (Davidson et al., 2015, J Virol 89:10982-92) was performed using HEK 293 cells expressing EBOV-GP with alanine (Ala) mutations at each position in the glycoprotein. First, protein mAb-11 or protein mAb-34 were run on the library to set up conditions and identify dropout mutations correlating with binding to GP. Serum from DMAb-11 or DMAb-34 administered mice were run using the same assay. For protein 5.6.1A2, drop out mutations I527A and W531A were pulled out utilizing the epitope mapping assay (FIG. 21A). The identical drop out mutations were identified for DMAb-11. For protein 15784, drop out mutations W531A, Y534A, F535A, and T565A were identified and the identical ones were observed for DMAb-34 (FIG. 21B). Representations of GP and binding sites are shown (FIG. 21, PDB 5JQ3, Zhao et al 2016).

Protection Against Ebola Virus in a Mouse Challenge Model

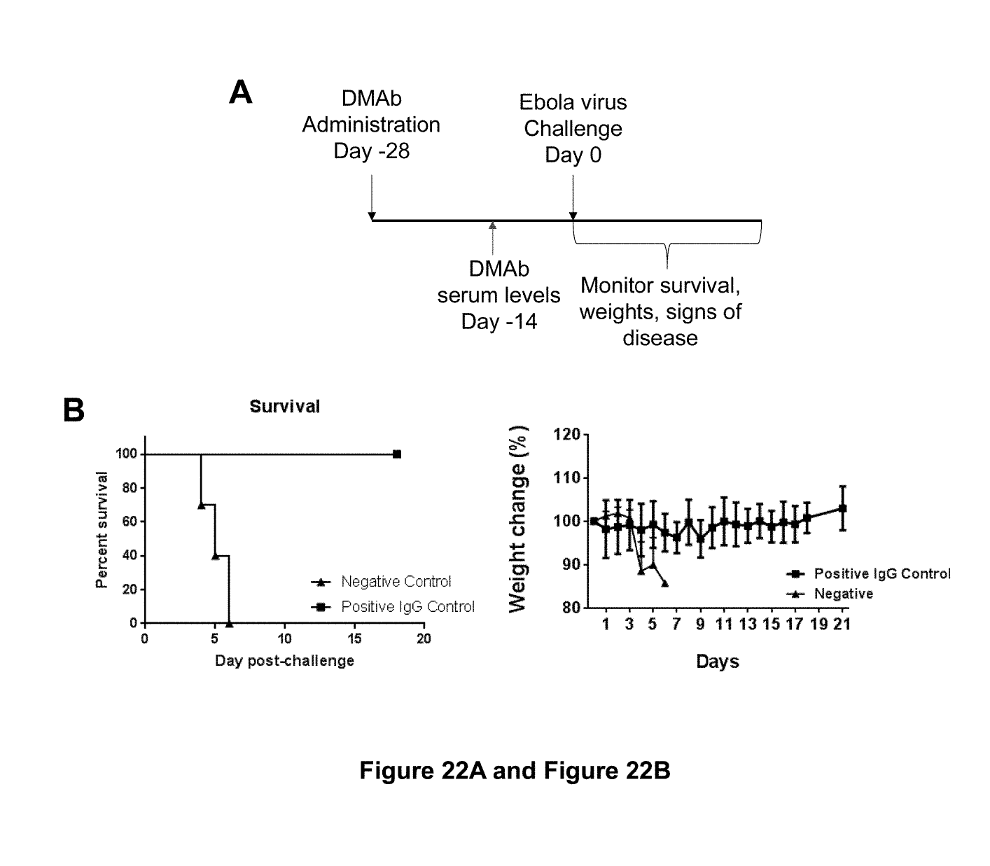

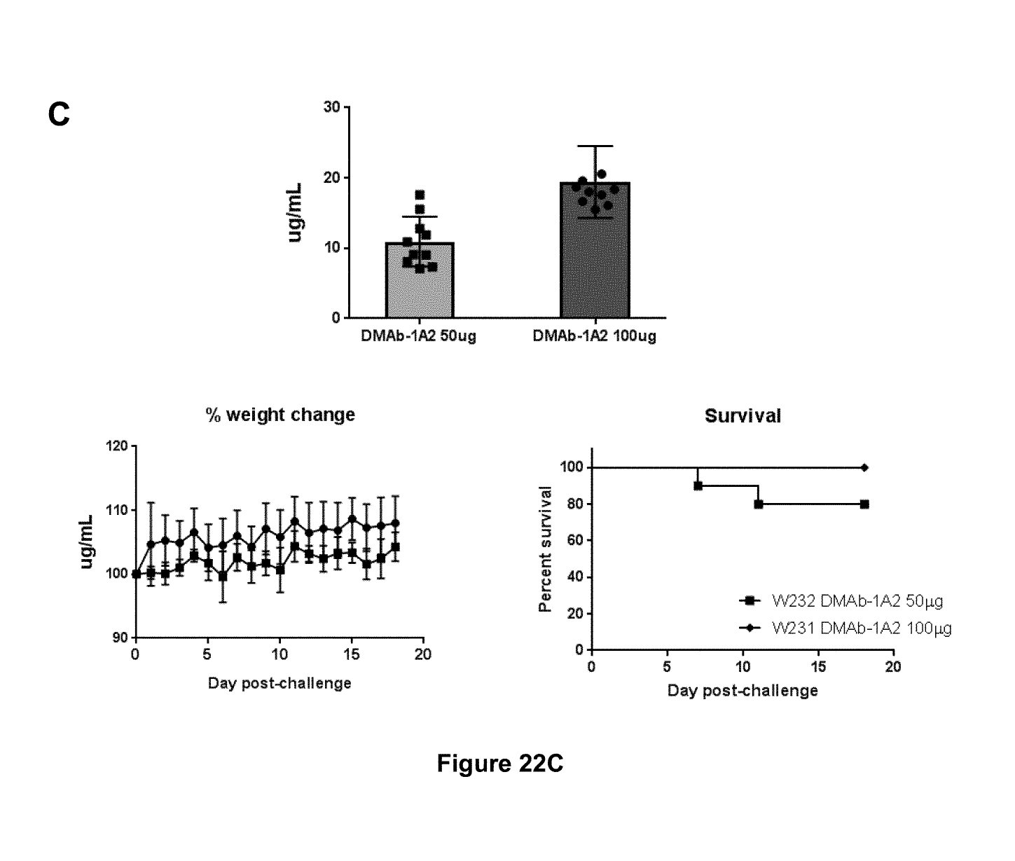

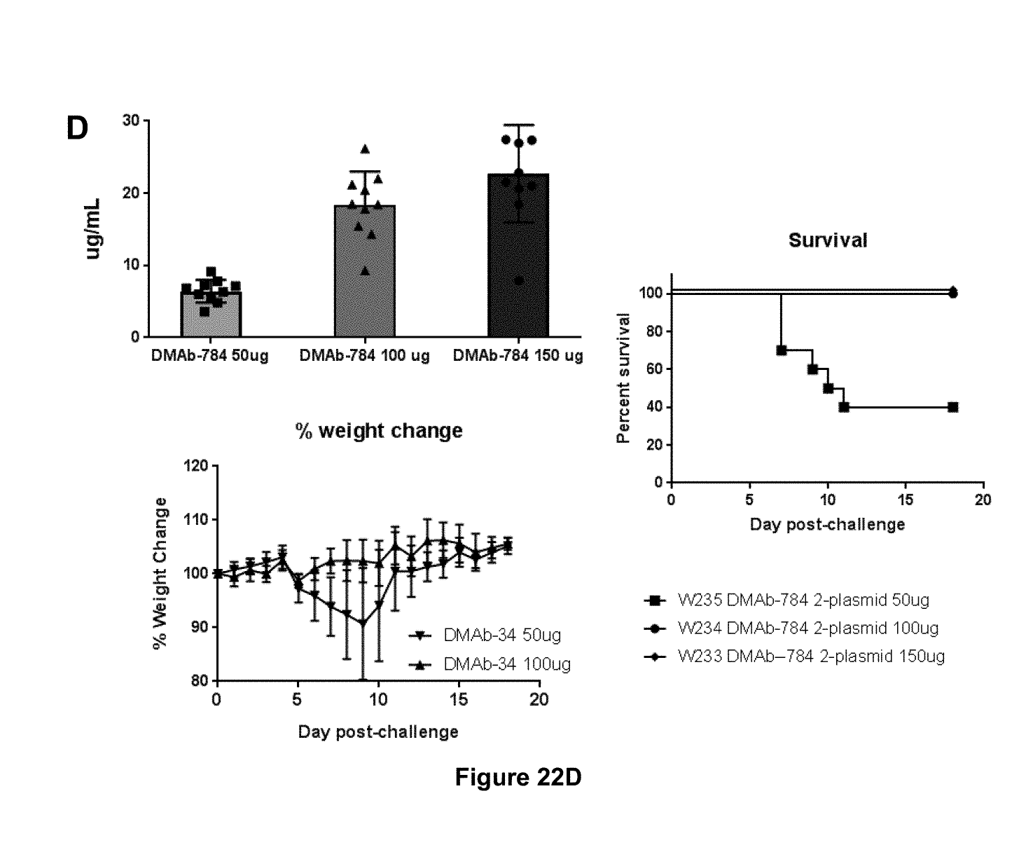

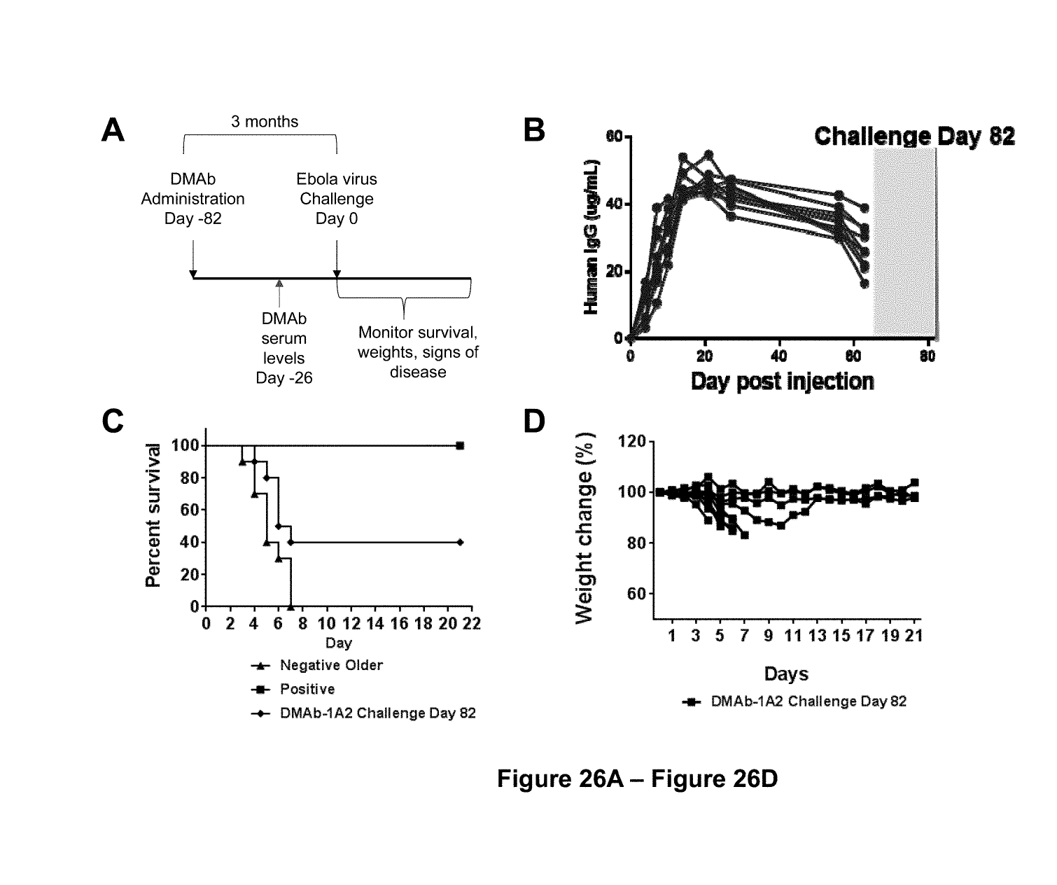

Doses of GP-DMAb-11 and DMAb-34 were administered to BALB/c mice at Day −28. On day −14, serum was harvested from animals before they were shipped to the biosafety level 4 (BSL4) containment laboratory at the Public Health Agency of Canada (PHAC, Winnipeg, Manitoba, Canada). DMAb-injected mice received 1000 times the median lethal dose (1000LD₅₀) challenge of mouse-adapted EBOV (strain Mayinga) on Day 0 (FIG. 22A). Negative control group (n=10) and positive protein IgG (n=10) groups were included (FIG. 22B). DMAb expression levels increased in a dose-dependent manner (FIGS. 22C and 22D). DMAb-11 was 100% protective at the 100 μg dose and high, 80% protection was observed with the lower 50 μg dose. No signs of disease were observed in surviving animals. 100% protection was observed with both the 100 μg and 150 μg doses of DMAb-34. A break in DMAb-34 protection was observed at the 50 μg dose, where only 40% of animals survived. To demonstrate that a single-plasmid is also protective, the one-plasmid construct was administered in different doses to BALB/c mice (FIG. 26). Animals received 200 μg, 300 μg, or 400 μg of total DMAb-11 single-plasmid DNA. High levels of protection (90-100% and no signs of morbidity) were observed with each of the three doses. The one-plasmid DMAb-11 was not tittered down with lower doses, however the data demonstrates that a one-plasmid DMAb construct is protective.

DMAb Co-Delivery

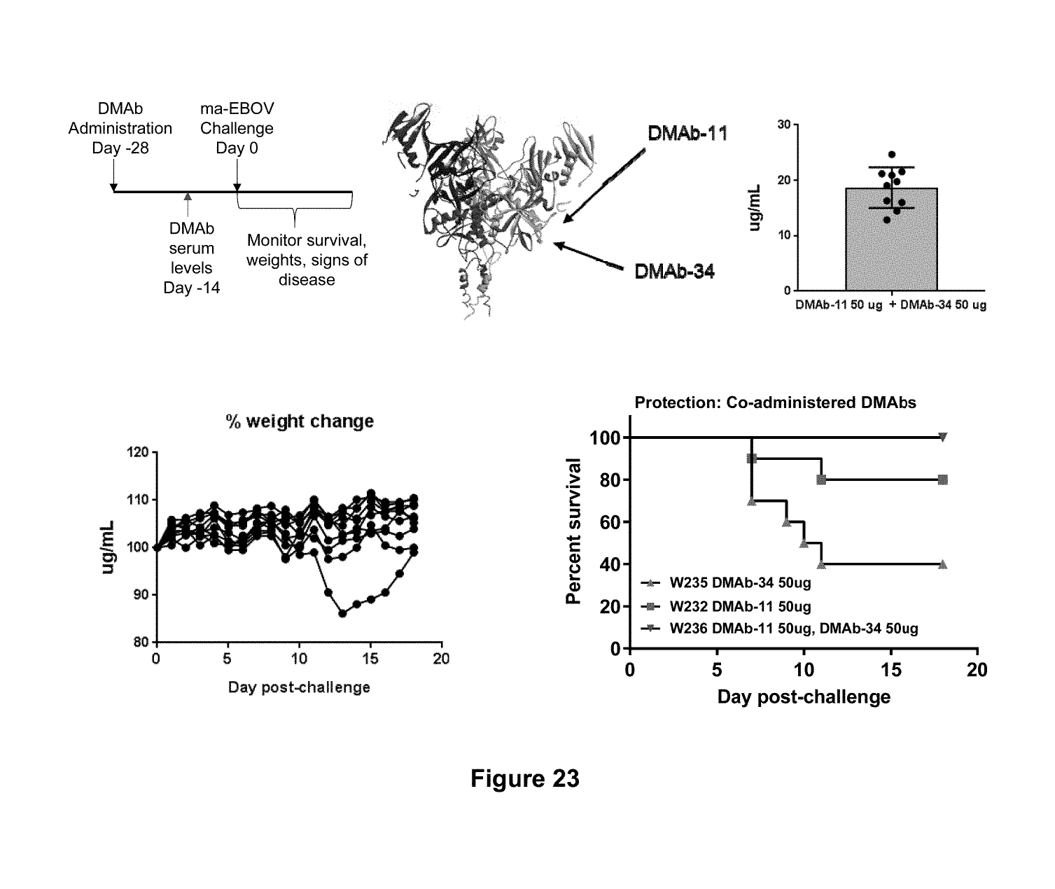

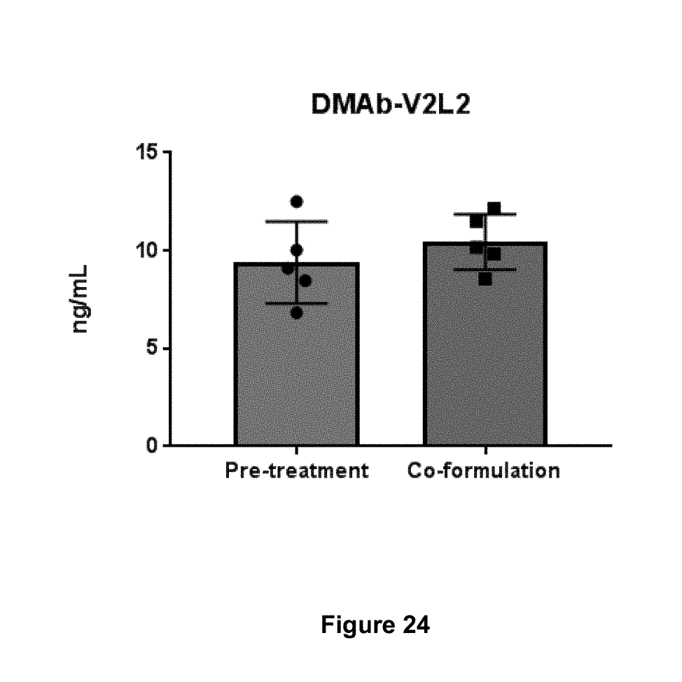

To address the concern of potential pathogen escape (Kugelman et al., 2015, Cell Rep 12:2111-20; Miler et al., 2016, Peer J 6:e1674), one strategy is co-delivery of more than one antibody clone targeting different epitopes. In the same experiment, both DMAb-11 and DMAb-34 were also co-delivered at separate injections sites on the mouse leg. Animals received 50 μg DMAb-11 in one hind limb and 50 μg DMAb-34 in the opposite hind limb. Total IgG (both DMAb-11 and DMAb-34) was assayed (FIG. 23). Animals were challenged on Day 28. 100% protection was observed with no signs of disease (FIG. 23C). One animal unexpectedly lost weight late during challenge however this animal fully recovered. The 50 μg dose groups of DMAb-11 and DMAb-34 from FIG. 22 are duplicated on this graph for ease of comparison.

Long-Term Protection

In one set of animals (n=10), DMAb-11 was administered and animals were challenged 82 days following initial DMAb administration. These animals receive a single-plasmid DMAb-construct. Serum levels were monitored on day 56 (day −26 before challenge) before the animals were shipped for challenge. Based on previous data (FIG. 18) it is likely that the animals had levels below 10 ug/mL at the time of challenge. Remarkably, 40% survival in these animals was observed suggesting that DMAbs can afford long-term protection (FIG. 26). This would be particularly beneficial during a vaccination regimen that requires multiple boosts in order to achieve optimal efficacy and supports evaluation of a potential co-administration approach with DMAb and vaccine.

### DISCUSSION

There are significant challenges for the uptake of mAb technologies to treat viral hemorrhagic fever (VHF) viruses. In vivo plasmid DNA delivery of genes encoding highly potent anti-Ebola virus GP mAbs could have a tremendous impact on prevention of infectious diseases like EVD. The DMAb approach fills an important gap between antibody production and in vivo administration, utilizing many of the discovery and technology advancements established by traditional mAb development. The field of bioprocessed IgG production has developed highly sophisticated in silico analysis (Seeliger et al., 2015, Mabs-Austin 7:505-15; Sharma et al., 2014, PNAS 111:18601-6), cell-line based large-scale bioreactors, and refined purification processes. It is demonstrated herein that, similarly, consistent expression of DMAbs in vivo also requires significant in silico sequence design, delivery, and formulation modifications to increase systemic human IgG expression. The efficacy this approach is supported by complete protection observed in mouse-adapted EBOV challenge experiments.

During the 2014-2016 EVD epidemic, an alternative mAb bioprocessing method utilizing Nicotiana benthamiana (tobacco) plants was used to produce the experimental ZMapp cocktail mAbs (Chen et al., 2016, F1000Res pii:912). ZMapp was administered as three intravenous infusions of 50 mg/kg, every three days (PREVAIL II WG et al., 2016, NEJM 375(15):1448-56). Considerable effort and resources went to rapid production of ZMapp cocktail, yet the high dose/patient and number of infusions draws attention to the need for novel strategies to effectively deliver potent mAbs in vivo. MAb protein stability during transport and cold-chain storage are additional hurdles for delivery of anti-GP protein biologics in resource-limited settings such as field clinics and developing countries. In vivo DNA-delivery strategies such as DMAb are potentially enabling for mAb administration utilizing a platform that is safe, non-integrating, and temperature-stable in a diverse range of environments. DMAbs are simple to modify as newer, highly potent mAb clones are identified. Importantly, DMAbs are an important research tool for quick investigation of mAbs targeting Ebola virus and other infectious disease pathogens. The studies described herein can be adapted to greatly expedite the simultaneous evaluation of multiple mAb clones in vivo.

The current study demonstrates that it is possible to study protective efficacy of human IgG DMAbs in mice. Mouse studies of bioprocessed mAb and in vivo vector-delivered mAb are hindered by the development of murine anti-human antibody immune responses. One approach is to convert human Fc to mouse Fc Ig, retaining the antigen-binding Fab, in an effort to minimize host anti-antibody responses. Not surprisingly, fully mouse IgG2a DNA-encoded mAbs exhibit long-term expression in mouse models and protect against lethal ma-EBOV challenge (Andrews et al., 2017, Mol Ther Methods Clin Dev 7:74-82). Although Fc conversion may afford experimental advantages to mouse IgG mAbs through preferential binding to mouse Fc gamma receptors, translation of murine DNA-encoded Ig candidates to humans is not trivial. As these studies demonstrate, amino acid changes can have significant impact on in vivo expression levels (FIG. 18) and reversion to a human Fc would likely have direct consequences on gene expression. Others have shown that mouse-human chimeric Ig and humanized mouse Fab VH and VL regions may significantly alter expression, binding, ultimately impacting protection against lethal ma-EBOV challenge (Limberis et al., 2016, J Infect Dis 214:1975-9). Altered antibody paratope binding and functionality has been observed with murine mAbs containing identical variable regions but different Fc isotypes (Janda et al., 2016, Front Microbiol 7:22; Janda et al., 2012, JBC 287:35409-17;), suggesting that the Fc domain may also place physical constraints on Fab allosteric cooperativity (Janda et al., 2016, Front Microbiol 7:22; Yang et al., 2017 Mabs-Austin 9:1231-52) with a potential impact on epitope specificity and virus neutralization virus neutralization (Tudor et al., 2012, PNAS 109:12680-5). In this context, the current anti-GP DMAb approach provides an important stepping stone for evaluation of human DMAb expression and protective efficacy that will likely be enhanced in NHPs and humans with matched antibody-receptor interactions and functional responses.

These studies demonstrate that anti-GP DMAbs can express for months, enabling potential administration with immunization campaigns to provide early protection during the time it takes to establish vaccine-induced memory responses. These studies provide an important foundation for DMAb development and translation of anti-GP DMAbs to larger animal models. Overall, the anti-GP DMAb approach provides a simple, transient in vivo delivery strategy for highly potent anti-EVD mAb clones. This approach can be applied to the engineering and screening of pan-filovirus DMAbs and clones targeting other infectious diseases. DMAbs can be easily administered to various demographic populations including deployable personnel, populations that are contraindicated for other treatments, and those living and working in resource-limited settings.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A nucleic acid molecule encoding one or more synthetic antibodies, wherein the nucleic acid molecule comprises at least one selected from the group consisting of
a) a nucleotide sequence encoding an anti-Ebola glycoprotein (GP) protein synthetic antibody;
b) a nucleotide sequence encoding a fragment of an anti-Ebola GP synthetic antibody.

2. The nucleic acid molecule of claim 1, further comprising a nucleotide sequence encoding a cleavage domain.

3. The nucleic acid molecule of claim 1, comprising a nucleotide sequence encoding an anti-Ebola GP antibody.

4. The nucleic acid molecule of claim 3, comprising a nucleotide sequence encoding one or more sequences selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, and SEQ ID NO:25.

5. The nucleic acid molecule of claim 3, comprising a nucleotide sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO: 16, SEQ ID NO:18, SEQ ID NO:20, SEQ ID NO:22, SEQ ID NO:24, and SEQ ID NO:26.

6. The nucleic acid molecule of claim 1, wherein the nucleic acid molecule comprises a nucleotide sequence having at least about 95% identity over an entire length of the nucleic acid sequence to a nucleic acid encoding a sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, and SEQ ID NO:25.

7. The nucleic acid molecule of claim 1, wherein the nucleic acid molecule comprises a nucleotide sequence having at least about 95% identity over an entire length of the nucleic acid sequence to a nucleic acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO: 16, SEQ ID NO:18, SEQ ID NO:20, SEQ ID NO:22, SEQ ID NO:24, and SEQ ID NO:26.

8. The nucleic acid molecule of claim 1, wherein the nucleotide sequence encodes a leader sequence.

9. The nucleic acid molecule of claim 1, wherein the nucleic acid molecule comprises an expression vector.

10. A composition comprising the nucleic acid molecule of claim 1.

11. The composition of claim 10, further comprising a pharmaceutically acceptable excipient.

12. A method of preventing or treating a disease in a subject, the method comprising administering to the subject the nucleic acid molecule of claim 1.

13. The method of claim 12, wherein the disease is an Ebola virus infection.

14. A method of preventing or treating a disease in a subject, the method comprising administering to the subject a composition of claim 10.
